(19) United States
(12) Patent Application Publication
(10) Pub. No.: US 2025/0137963 A1
(43) Pub. Date: May 1, 2025

(54) GAS SENSOR

(71) Applicant: NGK INSULATORS, LTD., Nagoya-shi (JP)

(72) Inventor: Shintaro MAKI, Nagoya-shi (JP)

(21) Appl. No.: 18/926,473

(22) Filed: Oct. 25, 2024

(30) Foreign Application Priority Data
Nov. 1, 2023 (JP) 2023-187624

(51) Int. Cl. G01N 27/407; G01N 33/00

(52) U.S. Cl. CPC G01N 27/4077; G01N 27/4078; G01N 33/0037

(57) ABSTRACT

The ceramic housing is prevented from moving to the distal end side in the axial direction while suppressing the risk of breakage of the members constituting the gas sensor. A gas sensor according to one aspect of the present invention includes a hollow member disposed between a main metal fitting and a ceramic housing, and the hollow member is made of a material having a Young's modulus in a range from 193 to 206 GPa.

### CROSS-REFERENCE TO RELATED APPLICATION

The present application claims priority from Japanese application JP 2023-187624, filed on Nov. 1, 2023, the contents of which is hereby incorporated by reference into this application.

### FIELD OF INVENTION

The present invention relates to a gas sensor.

### BACKGROUND

A gas sensor that detects a concentration of a specific gas such as oxygen or NOx in a measured gas such as an exhaust gas of an automobile is conventionally known. For example, JP 2022-173747A discloses a gas sensor including a sensor element, a main metal fitting surrounding the sensor element, a metal outer tube attached to a portion on a rear end side of the main metal fitting, and a rubber cap that closes a rear end side opening of the outer tube. In the gas sensor of JP 2022-173747A, an electrode terminal portion disposed on a rear end side of a sensor element is connected to a distal end side connecting portion of a terminal metal fitting, and the distal end side connecting portion and a portion of the sensor element having the electrode terminal portion are accommodated in a separator (ceramic housing). The separator is disposed inside the outer tube in a state of being sandwiched and fixed between the rubber cap and a distal end side member positioned on the distal end side with respect to the separator.

### SUMMARY OF INVENTION

The present inventors have found a problem that a conventional gas sensor as disclosed in JP 2022-173747A has a high risk of breakage of a member constituting the gas sensor.

That is, the conventional gas sensor prevents the separator from moving in the axial direction of the gas sensor by sandwiching the separator between the rubber cap and the distal end side member. In particular, in the conventional gas sensor, the distal end side member prevents the separator from moving to the distal end side of the gas sensor.

Therefore, the distal end side member extends toward the rear end side from the main metal fitting surrounding the sensor element so as to be in contact with the separator, and, for example, extends longer toward the rear end side from the main metal fitting. As described above, since the sensor element is connected to the terminal metal fitting inside the separator, the sensor element penetrates the inside of the distal end side member located on the distal end side with respect to the separator in the axial direction. That is, in the conventional gas sensor, the sensor element axially penetrates the inside of the distal end side member extending in the axial direction (for example, it extends longer toward the rear end side than the main metal fitting). Therefore, in the conventional gas sensor, for example, when the distal end side member is inclined from the axial direction, the sensor element extending in the axial direction and the distal end side member interfere with each other, and element breakage or the like may occur.

Further, since the distal end side member and the separator are in contact with each other in the conventional gas sensor, breakage or the like of a contact portion between the distal end side member and the separator may occur when an impact is applied to the gas sensor. In particular, in a case where the distal end side member and the separator are formed of the similar material, for example in a case where both are formed of ceramics such as alumina, the likelihood of occurrence of breakage or the like of the contact portion described above is further increased.

An aspect of the present invention has been made in view of such circumstances, and an object of the present invention is to provide a gas sensor that prevents a ceramic housing from moving to a distal end side in an axial direction while suppressing a risk of breakage of a member constituting the gas sensor.

In order to solve the above-described problem, the present invention adopts the following configuration.

A gas sensor according to a first aspect includes: a sensor element extending in an axial direction and having a connector electrode on a rear end side; a tubular main metal fitting in which the sensor element penetrates the inside in the axial direction; a tubular outer tube extending in the axial direction and attached to an outer peripheral surface on a rear end side of the main metal fitting; a terminal metal fitting extending in the axial direction and including an element contact portion electrically connected to the connector electrode on a distal end side; a ceramic housing accommodating the connector electrode and the element contact portion and disposed inside the outer tube; an elastic body disposed to seal an open end on a rear end side of the outer tube; and a hollow member including a material having a Young's modulus in a range from 193 to 206 GPa and disposed inside the outer tube, in which the sensor element penetrates the inside in the axial direction, a distal end side in the axial direction is in contact with the rear end side of the main metal fitting, and a rear end side in the axial direction is in contact with a distal end side of the ceramic housing.

With this configuration, in the gas sensor, the hollow member is in contact with the rear end side of the main metal fitting on the distal end side in the axial direction, that is, movement of the hollow member toward the distal end side is restricted by the main metal fitting. The ceramic housing is in contact with the hollow member restricted in movement to the distal end side. Therefore, in the gas sensor, the movement of the ceramic housing to the distal end side is restricted by the hollow member, that is, the hollow member prevents the ceramic housing from moving to the distal end side.

In the gas sensor, a material constituting the hollow member has a Young's modulus in a range from 193 to 206 GPa.

The present inventors have considered that the occurrence of damage or the like in the ceramic housing in contact with the hollow member can be suppressed by the hollow member being sufficiently soft. In addition, the present inventors have considered that, due to the hollow member being sufficiently hard, the hollow member can reliably (stably) prevent the movement of the ceramic housing toward the distal end side. Therefore, the present inventors conducted an experiment to verify the mechanical characteristics and the like of the material constituting the hollow member, which can suppress the occurrence of damage and the like in the ceramic housing and can reliably prevent the movement of the ceramic housing to the distal end side. As a result, the present inventors have confirmed that the above two requirements can be satisfied by setting the Young's modulus of the material constituting the hollow member to a range from 193 to 206 GPa. That is, the present inventors confirmed that by setting the Young's modulus of the material constituting the hollow member to a range from 193 to 206 GPa, it is possible to reliably prevent the movement of the ceramic housing to the distal end side while remarkably suppressing the occurrence of damage and the like in the ceramic housing.

Therefore, the hollow member formed of a material having a Young's modulus in a range from 193 to 206 GPa can reliably prevent movement of the ceramic housing toward the distal end side while remarkably suppressing occurrence of damage or the like in the ceramic housing.

Therefore, the gas sensor has an effect of preventing the ceramic housing from moving toward the distal end side in the axial direction while suppressing the risk of breakage of a member constituting the gas sensor.

A gas sensor according to a second aspect may be the gas sensor according to the first aspect, in which the hollow member is formed of a thin plate having a thickness in a range from 0.3 to 0.8 mm. In this configuration, in the gas sensor, the hollow member is formed of a thin plate having a thickness in a range from 0.3 to 0.8 mm.

Here, a heat source exists on the distal end side of the gas sensor, and thus, there is a possibility that heat generated by the heat source is transferred to the ceramic housing via the hollow member and further to the elastic body via the ceramic housing. When heat is transferred to the ceramic housing, for example, the contact between the connector electrode and the element contact portion is heated to a high temperature, whereby the contact is oxidized, and the contact resistance may increase. In addition, when heat is transferred to the elastic body, the elastic body may be eroded. When the elastic body is eroded, the reference gas (reference air) in the outer tube may be contaminated or the seismic resistance of the gas sensor may deteriorate. In addition, an electrode (for example, a reference electrode provided so as to be contactable with a reference gas) included in the sensor element may be contaminated by an organic gas accompanying decomposition of the elastic body by heat.

Therefore, the present inventors have studied suppressing heat transfer to the ceramic housing while maintaining the strength of the hollow member that prevents the ceramic housing from moving to the distal end side in the axial direction by devising the configuration of the hollow member. The present inventors have conducted an experiment and confirmed that the hollow member formed of a thin plate having a plate thickness in a range from 0.3 to 0.8 mm can remarkably suppress heat transfer to the ceramic housing while maintaining the strength of the hollow member. The present inventors have confirmed that the thin plate having a plate thickness in a range from 0.3 to 0.8 mm has sufficient strength to prevent the ceramic housing from moving to the distal end side in the axial direction and has sufficient heat capacity to suppress heat transfer to the ceramic housing.

Therefore, the hollow member formed of a thin plate having a plate thickness in a range from 0.3 to 0.8 mm can remarkably suppress heat transfer to the ceramic housing while having sufficient strength to prevent the ceramic housing from moving to the distal end side in the axial direction.

Therefore, the gas sensor can prevent the ceramic housing from moving to the distal end side in the axial direction while suppressing a risk of breakage of a member constituting the gas sensor, and can further improve heat resistance of the gas sensor.

A gas sensor according to a third aspect may be the gas sensor according to the first or second aspect, in which the hollow member includes a plurality of members. With this configuration, in the gas sensor, the hollow member includes the plurality of members, that is, the hollow member formed of a material having a Young's modulus in a range from 193 to 206 GPa includes the plurality of members. For example, the hollow member includes the plurality of members, and each of the plurality of members is formed of a material having a Young's modulus in a range from 193 to 206 GPa. Therefore, in the gas sensor, the hollow member formed of a material having a Young's modulus in a range from 193 to 206 GPa can be easily made of the plurality of members.

A gas sensor according to a fourth aspect may be the gas sensor according to any one of the first to third aspects, in which the hollow member and the main metal fitting are integrally formed. In this configuration, in the gas sensor, the hollow member and the main metal fitting are integrally formed. Therefore, the gas sensor has an effect that the hollow member that prevents the ceramic housing from moving to the distal end side in the axial direction can be easily configured together with the main metal fitting while suppressing the risk of breakage of the member constituting the gas sensor. The gas sensor has also an effect of suppressing the number of members constituting the gas sensor.

A gas sensor according to a fifth aspect may be the gas sensor according to any one of the first to fourth aspects, in which the ceramic housing may be spaced apart from the elastic body. In this configuration, in the gas sensor, the ceramic housing is spaced apart from the elastic body, that is, not in contact with the elastic body. The gas sensor has the following effects by the elastic body being spaced apart from the ceramic housing disposed on the distal end side with respect to the elastic body in the axial direction. That is, the gas sensor can prevent heat generated from a heat source on a distal end side of the gas sensor from being transferred to the elastic body via the ceramic housing, and can prevent a situation such as erosion of the elastic body caused by the heat.

Note that, for example, in the gas sensor, the inner peripheral surface of the outer tube accommodating the ceramic housing therein may include a portion (locking portion) inclined from the axial direction, and as an example, may include a portion (locking portion) orthogonal to the axial direction. The ceramic housing may be restricted from moving to the rear end side in the axial direction by being in contact with the locking portion on the inner peripheral surface of the outer tube on the rear end side in the axial direction. Therefore, the gas sensor can prevent the ceramic housing from moving to the rear end side in the axial direction without bringing the ceramic housing into contact with the elastic body.

A gas sensor according to a sixth aspect may be the gas sensor according to any one of the first to fifth aspects, in which the main metal fitting may include a surface orthogonal to the axial direction on a rear end side, and the hollow member may be in contact with the surface orthogonal to the axial direction of the main metal fitting. With this configuration, in the gas sensor, the main metal fitting includes a surface orthogonal to the axial direction on a rear end side, and the hollow member is in contact with the surface orthogonal to the axial direction of the main metal fitting. Therefore, the gas sensor can easily and stably position the hollow member in the axial direction using a surface of the main metal fitting orthogonal to the axial direction, and particularly, has an effect of easily and stably restricting the movement of the hollow member toward the distal end side. The gas sensor can easily and stably position the hollow member in the axial direction to easily and stably position the ceramic housing restricted in movement to the distal end side by the hollow member in the axial direction.

A gas sensor according to a seventh aspect is the gas sensor according to any one of the first to sixth aspects, in which the hollow member may be spaced apart from the sensor element. In this configuration, in the gas sensor, the hollow member is spaced apart from the sensor element, that is, the hollow member and the sensor element are not in contact with each other. Therefore, the gas sensor has an effect of reducing a risk that the hollow member and the sensor element interfere with each other to cause element breakage or the like. For example, even when the hollow member is inclined from the axial direction and the like due to application of an external force to the gas sensor or the like, the hollow member is spaced apart from the sensor element and thus does not interfere with the sensor element.

The gas sensor according to an eighth aspect may be the gas sensor according to any one of the first to seventh aspects, further including a spacer disposed between the ceramic housing and the elastic body in the axial direction. With this configuration, in the gas sensor, the elastic body is disposed on the rear end side in the axial direction with respect to the ceramic housing and the spacer. Therefore, the gas sensor has an effect that the ceramic housing and the spacer effectively prevent the heat generated by the heat source on the distal end side of the gas sensor from being transferred to the elastic body. The spacer disposed on the distal end side with respect to the elastic body in the axial direction is preferably formed of a heat-resistant material. By forming the spacer of a heat-resistant material, it is possible to prevent a situation in which the spacer disposed on the distal end side with respect to the elastic body in the axial direction is eroded due to heat generated by the heat source.

Therefore, the present invention can provide the gas sensor that can prevent the ceramic housing from moving toward the distal end side in the axial direction while suppressing the risk of breakage of a member constituting the gas sensor.

### BRIEF DESCRIPTION OF THE DRAWINGS

FIG. 1 is a schematic cross-sectional view schematically illustrating an example of a configuration of a gas sensor according to an embodiment;

FIG. 2 is an enlarged cross-sectional view of a main part illustrating an arrangement state of a hollow member of the gas sensor illustrated in FIG. 1 in an enlarged manner;

FIG. 3 is a schematic cross-sectional view schematically illustrating an example of a configuration of a gas sensor according to a first modification; and

FIG. 4 is a schematic cross-sectional view schematically illustrating an example of a configuration of a gas sensor according to a second modification.

### DETAILED DESCRIPTION OF THE PREFERRED EMBODIMENTS

Hereinafter, an embodiment (hereinafter, also referred to as “the present embodiment”) according to one aspect of the present invention will be described with reference to the drawings. However, the present embodiment described below is merely an example of the present invention in all respects. It goes without saying that various improvements and modifications can be made without departing from the scope of the present invention. That is, in carrying out the present invention, a specific configuration according to the embodiment may be appropriately adopted.

### Configuration Example

### <Overall Outline of Gas Sensor>

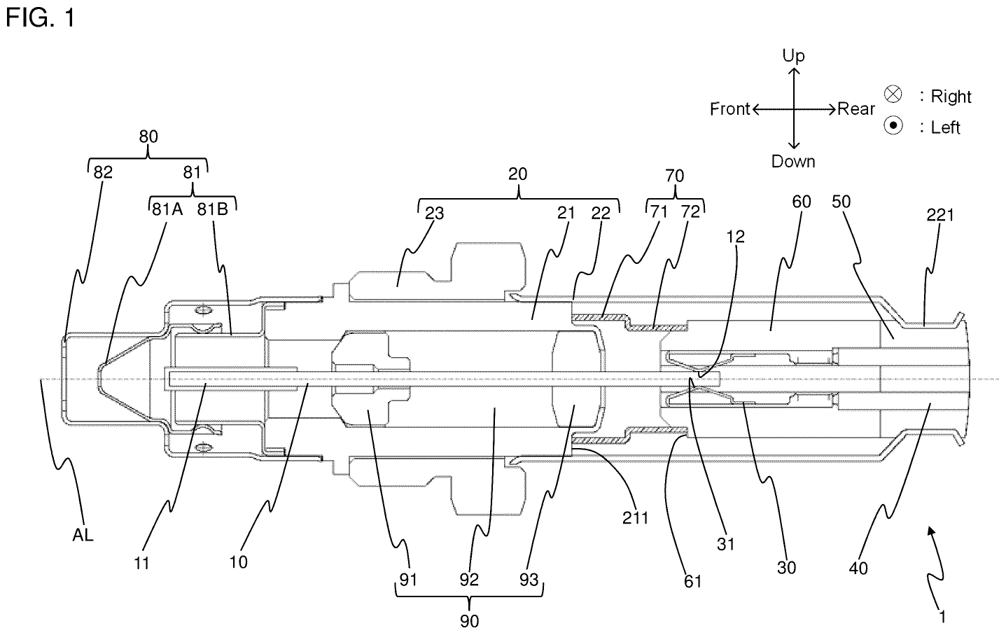

FIG. 1 is a schematic cross-sectional view schematically illustrating an example of a main configuration of a gas sensor 1 according to the present embodiment. That is, FIG. 1 schematically illustrates a configuration of a cross section of the gas sensor 1 parallel to and in contact with a longitudinal axis (axis AL, line along the left-right direction of the drawing). The gas sensor 1 is an example of a “gas sensor” of the present invention, and is capable of detecting a concentration (specific gas concentration) of a specific gas such as oxygen or NOx in a measured gas such as an exhaust gas of an automobile. As illustrated in FIG. 1, the gas sensor 1 has a shaft and is configured to extend along the longitudinal direction (axial direction), and has a distal end and a rear end as respective ends in the longitudinal direction. One end in the longitudinal direction is the distal end, and the other end is the rear end. In the example of FIG. 1, the gas sensor 1 is disposed such that the distal end thereof is directed to left and the rear end thereof is directed to right. That is, the left-right direction in FIG. 1 corresponds to the longitudinal direction (axial direction).

The gas sensor 1 according to the present embodiment includes at least a sensor element 10, a main metal fitting 21, an outer tube 22, a terminal metal fitting 30, an elastic body 50, a ceramic housing 60, and a hollow member 70. The gas sensor 1 illustrated in FIG. 1 further includes a fixing bolt 23, a lead wire 40, a protective cover 80, and an annular component 90. In the gas sensor 1, the sensor element 10 is surrounded by a tubular body 20 and the protective cover 80, and the tubular body 20 and the protective cover 80 constitute as a whole an accommodation member (casing) that accommodates the sensor element 10 therein. The sensor element 10 is disposed coaxially with the tubular body 20 and the protective cover 80, and the extending direction of the central axis of the sensor element 10 coincides with the axial direction of the gas sensor 1.

### (Sensor Element)

The sensor element 10 is an example of a “sensor element” of the present invention, and is configured to extend along the axial direction (left-right direction in FIG. 1). The sensor element 10 illustrated in FIG. 1 is an elongated flat plate-like (elongated plate-like) element. The sensor element 10 includes a detection unit 11 on the distal end side and a connector electrode 12 on the rear end side. The sensor element 10 exemplified in FIG. 1 has a distal end side coated with an outer porous layer, and the outer porous layer serves as a protective layer that suppresses occurrence of a crack in the element body of the sensor element 10 due to adhesion of moisture or the like in the measured gas, for example.

In the gas sensor 1, the sensor element 10 is disposed such that the distal end side faces the distal end of the gas sensor 1. For example, in one aspect of the sensor element 10, the measured gas introduced into the sensor element 10 is reduced or decomposed in the sensor element 10 to generate oxygen ions. In the gas sensor 1 including the sensor element 10, the concentration of the specific gas is obtained on the basis of the fact that the amount of oxygen ions flowing inside the sensor element 10 is proportional to the concentration of the specific gas that is the gas to be detected in the measured gas.

In the example illustrated in FIG. 1, the distal end side of the sensor element 10 is surrounded by a protective cover 80, the rear end side protrudes into the outer tube 22, and a substantially central portion between the distal end side and the rear end side is fixed inside the main metal fitting 21 by an annular component 90 in such a manner as to hermetically seal between both ends.

### (Annular Component)

In the example shown in FIG. 1, the annular component 90 includes a first ceramic supporter 91, a green compact 92, and a second ceramic supporter 93. The first ceramic supporter 91 and the second ceramic supporter 93 are ceramic insulators. More specifically, through holes (not shown) having shapes corresponding to the cross-sectional shape of the sensor element 10 are provided at axial center positions of the first ceramic supporter 91 and the second ceramic supporter 93, and the sensor element 10 is inserted into the through holes, whereby the first ceramic supporter 91 and the second ceramic supporter 93 are annularly mounted on the sensor element 10. The first ceramic supporter 91 is locked to the tapered surface of the main metal fitting 21 on the left side of the drawing.

On the other hand, the green compact 92 is obtained by molding a ceramic powder such as talc. In the green compact 92, as with the first ceramic supporter 91 and the second ceramic supporter 93, the sensor element 10 is inserted into the through hole, so that two molded bodies (not shown) annularly mounted on the sensor element 10 are disposed inside the main metal fitting 21 in a state of being annularly mounted around the sensor element 10, and then further compressed and integrated. More specifically, the ceramic particles constituting the green compact 92 are surrounded by the first ceramic supporter 91, the second ceramic supporter 93 and the main metal fitting 21, and a space through which the sensor element 10 penetrates inside the main metal fitting 21 is closely filled with the particles. The compressed filling of the green compact 92 achieves hermetic sealing between the distal end side and the rear end side of the sensor element 10.

FIG. 1 shows an example in which the annular component 90 includes the first ceramic supporter 91, the green compact 92, and the second ceramic supporter 93. However, in the gas sensor 1, it is not essential that the annular component 90 be composed of the first ceramic supporter 91, the green compact 92, and the second ceramic supporter 93. The gas sensor 1 illustrated in FIG. 1 includes the annular component 90 that fixes the sensor element 10 inside the main metal fitting 21 and hermetically seals between the distal end side and the rear end side of the sensor element 10.

### (Tubular Body)

The tubular body 20 is, for example, a tubular (for example, cylindrical) member formed of metal, and has an open end. The sensor element 10 is disposed inside the tubular body 20. In the example shown in FIG. 1, the tubular body 20 includes the tubular main metal fitting 21, the tubular outer tube 22, and the fixing bolt 23, each of which is a metal member.

The main metal fitting 21 is an example of the “main metal fitting” of the present invention, and is, for example, a tubular (for example, cylindrical) member formed of metal. Inside the main metal fitting 21, the sensor element 10 and the annular component 90 for fixing, which is annularly mounted on the sensor element 10, are accommodated. That is, the main metal fitting 21 is further annularly mounted around the annular component 90 annularly mounted around the sensor element 10. The main metal fitting 21 illustrated in FIG. 1 is configured to surround the sensor element 10 along the axial direction (longitudinal direction), and is particularly configured to surround a range excluding a part of each of the distal end side and the rear end side of the sensor element 10. That is, the main metal fitting 21 is a tubular member in which the sensor element 10 penetrates the inside in the axial direction, and surrounds the periphery of the sensor element 10, in particular, the periphery of the sensor element 10 except for a part of each of the distal end side and the rear end side of the sensor element 10.

The outer tube 22 is an example of the “outer tube” of the present invention, is a tubular (for example, cylindrical) member extending in the axial direction, and is formed of metal, for example. The outer tube 22 exemplified in FIG. 1 covers the rear end of the sensor element 10, the hollow member 70, and the periphery of the ceramic housing 60 (terminal metal fitting 30).

The outer tube 22 is attached to a portion on the rear end side of the main metal fitting 21, and is attached to, for example, an outer peripheral surface on the rear end side of the main metal fitting 21. In the example illustrated in FIG. 1, the end portion (open end) on the distal end side of the outer tube 22 is attached to the outer peripheral end portion on the rear end side of the main metal fitting 21, and is, for example, welded and fixed to the outer peripheral end portion on the rear end side of the main metal fitting 21. In addition, the elastic body 50 is disposed at the open end on the rear end side of the outer tube 22 so as to seal the open end. On the rear end side of the outer tube 22, a reduced diameter portion 221 is formed for swaging a part of the elastic body 50 to seal the open end on the rear end side from the periphery. In the reduced diameter portion 221, the outer tube 22 is swaged from the outside in a diameter-reducing manner over the entire circumferential direction thereof, so that a reaction force directed radially outward is generated in the elastic body 50, whereby the outer tube 22 is sealed.

In addition, the lead wire 40 is drawn out from the open end on the rear end side of the outer tube 22 sealed by the elastic body 50 to the outside through a through hole (not illustrated) formed inside the elastic body 50. The internal space of the outer tube 22 has a reference gas (atmospheric air) atmosphere, and the rear end of the sensor element 10 is disposed in the internal space of the outer tube 22 filled with the reference gas. For example, the external air (atmosphere) may be introduced into the internal space of the outer tube 22 through between the coating of the lead wire 40 and the metal wire (conductor) (in other words, the inside of the coating), so that the internal space of the outer tube 22 becomes the reference gas (atmospheric air) atmosphere. However, the configuration for introducing the reference gas into the internal space of the outer tube 22 is not limited to the above-described configuration, and the reference gas may be introduced into the internal space of the outer tube 22 through a reference gas introduction path (reference gas introduction hole) (not illustrated) other than “between the coating of the lead wire 40 and the metal wire”.

The fixing bolt 23 is an annular member used to fix the gas sensor 1 to the measurement position (attachment position), and is fixed coaxially with the main metal fitting 21. The fixing bolt 23 includes a threaded bolt portion and a holding portion held when the bolt portion is being screwed. The bolt portion of the fixing bolt 23 is screwed with a nut provided at the attachment position of the gas sensor 1. For example, when a bolt portion of the fixing bolt 23 is screwed into a nut (nut portion) provided in an exhaust pipe of an automobile, the gas sensor 1 is fixed to the exhaust pipe in a mode in which the protective cover 80 side thereof is exposed to the inside of the exhaust pipe.

As described above, for example, the tubular body 20 (the main metal fitting 21, the outer tube 22, and the fixing bolt 23) and the gas sensor 1 (the sensor element 10) are coaxial, and the tubular body 20 has the distal end and the rear end as the respective ends in the axial direction (longitudinal direction). The tubular body 20 is disposed such that the distal end thereof faces the distal end of the gas sensor 1. Inside the tubular body 20, the sensor element 10, the fixing annular component 90 annularly mounted on the sensor element 10, the hollow member 70, and the ceramic housing 60 (terminal metal fitting 30) are accommodated, and the open end on the rear end side is sealed by the elastic body 50. The reduced diameter portion 221 for fixing the elastic body 50 to seal the open end of the tubular body 20 is formed on the rear end side of the tubular body 20 (outer tube 22), and the reduced diameter portion 221 swages a part of the elastic body 50 from the periphery.

In the gas sensor 1, the tubular body 20 does not necessarily include the fixing bolt 23, and the tubular body 20 may not include the fixing bolt 23. In the tubular body 20, the main metal fitting 21 and the outer tube 22 may be integrally formed. In the gas sensor 1, the tubular body 20 is only required to be a tubular member in which the sensor element 10 is disposed and an open end is formed.

### (Terminal Metal Fitting)

The terminal metal fitting 30 is an example of the “terminal metal fitting” of the present invention. The terminal metal fitting 30 is a metal member (contact member) extending in the axial direction. In the gas sensor 1, the sensor element 10 (in particular, the connector electrode 12 thereof) and the lead wire 40 are electrically connected via the terminal metal fitting 30. The terminal metal fitting 30 includes an element contact portion 31 electrically connected to the connector electrode 12 of the sensor element 10 on the distal end side, and a lead wire holding portion that crimps and holds the lead wire 40 on the rear end side.

### (Ceramic Housing)

The ceramic housing 60 is an example of the “ceramic housing” of the present invention, and is disposed (accommodated) inside the outer tube 22. The ceramic housing 60 may be configured as, for example, a tubular member extending in the axial direction, and as an example, may be configured as a cylindrical member extending in the axial direction. The ceramic housing 60 is a ceramic member that accommodates the rear end side (specifically, the connector electrode 12 provided on the rear end side of the sensor element 10) of the sensor element 10 and the element contact portion 31 of the terminal metal fitting 30. That is, in the gas sensor 1 exemplified in FIG. 1, the sensor element 10 (in particular, the connector electrode 12) and the terminal metal fitting 30 (in particular, the element contact portion 31) are electrically connected in the ceramic housing 60.

For example, the rear end side of the sensor element 10 provided with the connector electrode 12 is inserted into the ceramic housing 60 accommodating the distal end side (element contact portion 31) of the terminal metal fitting 30. In such an insertion state, the connector electrode 12 provided on the rear end side of the sensor element 10 and the distal end side (the element contact portion 31) of the terminal metal fitting 30 are in contact with each other. The distal end side (the element contact portion 31) of the terminal metal fitting 30 may be sandwiched and fixed between the rear end side of the sensor element 10 provided with the connector electrode 12 and the ceramic housing 60, so that the connector electrode 12 of the sensor element 10 and the terminal metal fitting 30 may be electrically connected.

In the gas sensor 1 exemplified in FIG. 1, there is a gap between the ceramic housing 60 and the tubular body 20 (outer tube 22), that is, the ceramic housing 60 is not in contact with the tubular body 20. That is, the ceramic housing 60 is disposed inside the outer tube 22 in a mode spaced apart from the inner peripheral surface of the outer tube 22. The ceramic housing 60 may be integrated with the sensor element 10, for example, so that the position (position in the radial direction) in the outer tube 22 is fixed without being in contact with the inner peripheral surface of the outer tube 22. As described above, the position of the sensor element 10 inside the tubular body 20 is fixed by the annular component 90 and the tubular body 20 (in particular, the main metal fitting 21 and the outer tube 22). Therefore, integrated with the sensor element 10 whose position in the tubular body 20 is fixed by the annular component 90 and the tubular body 20, the position of the ceramic housing 60 in the tubular body 20 may be fixed without being in contact with the tubular body 20. However, it is not essential for the gas sensor 1 to fix the position of the ceramic housing 60 inside the outer tube 22 in a state where the ceramic housing 60 and the inner peripheral surface of the outer tube 22 are spaced apart from each other by integrating the ceramic housing 60 and the sensor element 10.

### (Lead Wire)

The lead wire 40 is electrically connected to the connector electrode 12 of the sensor element 10 via the terminal metal fitting 30, and extends outward (for example, to the rear end side) from the open end of the tubular body 20. Specifically, the lead wire 40 is electrically connected to the rear end side (specifically, the lead wire holding portion of the terminal metal fitting 30) of the terminal metal fitting 30 on the distal end side thereof, and the rear end side of the lead wire 40 extends outward from the open end of the tubular body 20. As described above, the gap between the lead wire 40 and the tubular body 20 (the outer tube 22) is sealed by the elastic body 50.

For example, the lead wire 40 is inserted into a through hole (not illustrated) provided in the elastic body 50. The end portion on the distal end side of the lead wire 40 is crimped and fixed to the rear end side (the lead wire holding portion) of the terminal metal fitting 30, and the end portion on the rear end side of the lead wire 40 is connected to an external device (controller), a power supply, and the like. As a result, the sensor element 10 (in particular, the connector electrode 12 of the sensor element 10) is electrically connected to the external device, the power supply, and the like through the terminal metal fitting 30 and the lead wire 40. Although FIG. 1 illustrates an example in which there are two terminal metal fittings 30 and two lead wires 40, this is merely for the sake of simplicity of illustration. In practice, the gas sensor 1 includes the terminal metal fittings 30 and the lead wires 40 as many as necessary for the above-described electrical connection.

### (Elastic Body)

The elastic body 50 is an example of the “elastic body” of the present invention. The elastic body 50 is a member having elasticity, and is formed of, for example, rubber. The elastic body 50 is disposed so as to seal the open end (in the example illustrated in FIG. 1, the open end on the rear end side) of the tubular body 20, and is fixed to the outer tube 22 by the reduced diameter portion 221 in a mode of closing the open end (opening) on the rear end side of the outer tube 22, for example. In the example illustrated in FIG. 1, the lead wire 40 is inserted into the elastic body 50. Specifically, a through hole extending in the axial direction is formed inside the elastic body 50, and for example, a plurality of through holes extending in the axial direction is formed. The lead wire 40 is accommodated (inserted) in the through hole formed inside the elastic body 50, and for example, each of the plurality of lead wires 40 is accommodated (inserted) in each of the plurality of through holes formed inside the elastic body 50.

The material of the elastic body 50 is, for example, fluororubber. Fluororubber has excellent properties in various aspects such as resistance and strength, and is particularly excellent in heat resistance and oil resistance. Therefore, the gas sensor 1 uses the elastic body 50 formed of fluororubber, so that, for example, the sealing property of the elastic body 50 can be secured even in a high-temperature environment, and the detection accuracy of the gas concentration can be maintained and improved. However, it is not essential for the gas sensor 1 to use fluororubber as the material of the elastic body 50, and the gas sensor 1 may use an appropriate material having elasticity as the material of the elastic body 50.

### (Protective Cover)

The protective cover 80 is a substantially cylindrical exterior member that protects a predetermined range on the distal end side, which is a portion of the sensor element 10 that is directly in contact with the measured gas during use. The protective cover 80 illustrated in FIG. 1 is configured to surround at least a part of the distal end side of the tubular body 20 (main metal fitting 21) along the axial direction (longitudinal direction) and extend beyond the distal end of the sensor element 10. For example, the protective cover 80 is configured to surround the sensor element 10 and a part of the distal end side of the tubular body 20 around the axis. The protective cover 80 has a distal end and a rear end as respective ends in the axial direction, and the distal end of the protective cover 80 is disposed on the distal end side of the gas sensor 1 with respect to the distal end of the sensor element 10.

The protective cover 80 is provided with a plurality of through holes (not illustrated) through which gas can pass. The measured gas flowing into the protective cover 80 through the through hole is to be directly detected by the sensor element 10. The type, the number, the arrangement positions, the shape, and the like of the through-holes provided in the protective cover 80 may be appropriately determined in consideration of the inflow mode of the measured gas into the protective cover 80.

In the example illustrated in FIG. 1, the protective cover 80 includes a bottomed tubular inner cover 81 that covers the distal end of the sensor element 10 and a bottomed tubular outer cover 82 that covers the inner cover 81. The inner cover 81 includes a first member 81B and a second member 81A, and is configured to cover the periphery of at least a part of the distal end side of the sensor element 10 and the tubular body 20 (main metal fitting 21). The first member 81B extends along the axial direction from the outer wall of the distal end portion of the tubular body 20, and is configured to further extend along the axial direction, after the diameter decreases in the direction perpendicular to the axial direction beyond the distal end of the tubular body 20. The second member 81A is configured to cover the periphery of a part of the distal end side of the first member 81B. The outer cover 82 is configured to cover the periphery of the inner cover 81.

A sensor element chamber is formed as a space surrounded by the inner cover 81, and a distal end of the sensor element 10 is disposed in the sensor element chamber.

Openings are appropriately provided in the first member 81B, the second member 81A, and the outer cover 82 of the inner cover 81, whereby the sensor element chamber is connected to a space outside the protective cover 80. However, the configuration and shape of the protective cover 80 are not limited to such an example. The configuration and shape of the protective cover 80 may be appropriately determined according to the embodiment.

As a material of the protective cover 80, for example, a metal material such as stainless steel (for example, SUS) may be used. The protective cover 80 may be manufactured by appropriately molding the metal material. The protective cover 80 may be omitted from the configuration of the gas sensor 1.

### (Hollow Member)

The gas sensor 1 includes the hollow member 70 in order to prevent the ceramic housing 60 from moving to the distal end side in the axial direction while suppressing a risk of breakage of a member constituting the gas sensor 1. The hollow member 70 is an example of the “hollow member” of the present invention. The hollow member 70 exemplified in FIG. 1 includes, for example, two cylindrical portions (in the illustrated example, the first portion 71 and the second portion 72) having different diameters and arranged coaxially, and is configured as a cylindrical member having an outer diameter changing in the axial direction as a whole. That is, the hollow member 70 is a hollow member having a cylindrical (columnar) appearance. Specifically, the hollow member 70 includes the first portion 71 disposed on the distal end side in the axial direction, and the second portion 72 disposed on the rear end side of the first portion 71 in the axial direction and having a smaller diameter than the first portion 71. The outer diameter of the hollow member 70 changes in the axial direction. In the illustrated example, the outer diameter of the hollow member 70 changes at the connection portion between the first portion 71 and the second portion 72. Specifically, the outer diameter of the hollow member 70 decreases at the connection portion between the first portion 71 and the second portion 72.

In the hollow member according to one aspect of the present invention, it is not essential that the diameter of the first portion and the diameter of the second portion are different from each other, and the diameters of the first portion 71 and the second portion 72 may be the same. Further, it is not essential for the hollow member 70 that the second portion 72 has a smaller diameter than the first portion 71. Furthermore, in the example shown in FIG. 1, the outer diameter of the hollow member 70 varies at the connection portion between the first portion 71 and the second portion 72. However, the outer diameter of the hollow member 70 may change continuously or discontinuously from the opening on the distal end side (the opening on the distal end side of the first portion 71) to the opening on the rear end side (the opening on the rear end side of the second portion 72).

The first portion 71 is a cylindrical portion provided on the distal end side of the hollow member 70, and is in contact with the rear end side of the main metal fitting 21, and in the example illustrated in FIG. 1, is in contact with the rear end surface 211 provided on the rear end side of the main metal fitting 21. The second portion 72 is a cylindrical portion provided on the rear end side of the hollow member 70, is in contact with the distal end side of the ceramic housing 60, and is in contact with the first distal end surface 61 provided on the distal end side of the ceramic housing 60 in the example shown in FIG. 1.

In the gas sensor 1, the hollow member 70 is disposed inside the outer tube 22, and particularly, is disposed between the main metal fitting 21 and the ceramic housing 60 in the axial direction. Specifically, the hollow member 70 is disposed inside the outer tube 22 in a state where the distal end side (that is, the first portion 71) is in contact with the rear end side of the main metal fitting 21 and the rear end side (that is, the second portion 72) is in contact with the distal end side of the ceramic housing 60. The sensor element 10 penetrates the inside of the hollow member 70 in the axial direction, that is, the sensor element 10 penetrates the inside of the first portion 71 and the second portion 72 in the axial direction.

In the gas sensor 1, the hollow member 70 is in contact with the rear end side of the main metal fitting 21 on the distal end side in the axial direction, so that the movement of the hollow member 70 to the distal end side is restricted by the main metal fitting 21. Then, the ceramic housing 60 is in contact with the hollow member 70 on the distal end side thereof, that is, the hollow member 70 restricted in movement to the distal end side. Therefore, in the gas sensor 1, the movement of the ceramic housing 60 to the distal end side is restricted by the hollow member 70, that is, the hollow member 70 prevents the ceramic housing 60 from moving to the distal end side.

The hollow member 70 is formed of a material having a Young's modulus in a range from 193 to 206 GPa, for example, a metal plate (SUS430, SUS304, low carbon steel, etc.) having a Young's modulus in a range from 193 to 206 GPa.

Here, the present inventors have considered that occurrence of damage or the like in the ceramic housing 60 in contact with the hollow member can be suppressed by sufficiently softening the hollow member (for example, the hollow member 70) disposed between the main metal fitting 21 and the ceramic housing 60. In addition, the present inventors have considered that, due to the hollow member (for example, the hollow member 70) being sufficiently hard, the hollow member can reliably (stably) prevent the movement of the ceramic housing 60 toward the distal end side. Therefore, the present inventors conducted an experiment (first test described later) to verify the mechanical characteristics and the like of the material constituting the hollow member, which can suppress the occurrence of damage and the like in the ceramic housing 60 and can reliably prevent the movement of the ceramic housing 60 to the distal end side. As a result, the present inventors have confirmed that the above two requirements can be satisfied by setting the Young's modulus of the material constituting the hollow member to a range from 193 to 206 GPa. That is, the present inventors confirmed that by setting the Young's modulus of the material constituting the hollow member to 193 to 206 GPa, it is possible to reliably prevent the movement of the ceramic housing 60 to the distal end side while remarkably suppressing the occurrence of damage and the like in the ceramic housing 60.

The hollow member 70 is formed of a material having a Young's modulus in a range from 193 to 206 GPa, that is, the hollow member 70 is sufficiently soft at a contact portion with respect to the ceramic housing 60. Therefore, the hollow member 70 formed of a material having a Young's modulus in a range from 193 to 206 GPa can remarkably suppress the occurrence of breakage or the like in the ceramic housing 60, particularly in a contact portion of the ceramic housing 60 with respect to the hollow member 70. That is, the hollow member 70 formed of a material having a Young's modulus in a range from 193 to 206 GPa can remarkably suppress the occurrence of damage and the like at the contact portion of the ceramic housing 60 with respect to the hollow member 70. In the hollow member 70, due to the contact portion (second portion 72) with respect to the ceramic housing 60 being sufficiently soft, it is possible to prevent, for example, the occurrence of a situation in which “the ceramic housing 60 is damaged by collision with the hollow member 70”. In addition, the hollow member 70 formed of a material having a Young's modulus in a range from 193 to 206 GPa has sufficient hardness that can reliably (stably) prevent movement of the ceramic housing 60 toward the distal end side. That is, since the hollow member 70 is formed of a material having a Young's modulus in a range from 193 to 206 GPa, it is possible to reliably (stably) prevent the movement of the hollow member toward the distal end side of the ceramic housing 60 without being deformed or damaged. As described above, in the gas sensor 1, the hollow member 70 can prevent the ceramic housing 60 from moving to the distal end side in the axial direction while suppressing the risk of damaging the member (for example, the ceramic housing 60) constituting the gas sensor 1.

In the gas sensor 1, the hermetic sealing between the distal end side and the rear end side of the sensor element 10 is realized by the main metal fitting 21 (and the annular component 90), and the hollow member 70 disposed between the main metal fitting 21 and the ceramic housing 60 does not get involved in (affect) the hermetic sealing. That is, in the gas sensor 1, the hermetic sealing described above and the restriction of the movement of the ceramic housing 60 toward the distal end side are realized by different members. Specifically, the former is realized by the main metal fitting 21 (and the annular component 90), while the latter is realized by the hollow member 70.

Here, in the conventional gas sensor disclosed in JP 2022-173747A, the distal end side member attempts to achieve both hermetic sealing between the distal end side and the rear end side of the sensor element and restriction of movement of the ceramic housing (separator) toward the distal end. However, for example, when the distal end side member is strongly pushed toward the distal end side in order to realize hermetic sealing between the distal end side and the rear end side of the sensor element, the distal end side member moves toward the distal end side. When the distal end side member moves toward the distal end, restriction of movement of the distal end side member to the distal end of the ceramic housing (fixing force with respect to the ceramic housing) becomes insufficient, and a problem such as deterioration of vibration resistance performance of the gas sensor occurs. That is, in the conventional gas sensor, since both the hermetic sealing and the movement restriction described above are achieved by one member (specifically, the distal end side member), it is difficult to achieve the former while sufficiently achieving the latter, for example.

On the other hand, in the gas sensor 1, (1) hermetic sealing between the distal end side and the rear end side of the sensor element is realized by the main metal fitting 21 (and the annular component 90), and (2) restriction of movement to the distal end side of the ceramic housing 60 is realized by the hollow member 70. Therefore, the gas sensor 1 can achieve both the hermetic sealing and the movement restriction described above without causing a problem that a conventional gas sensor has, such as “in order to realize the above-mentioned hermetic sealing, it is difficult to realize the movement restriction”.

In the hollow member 70, each of the first portion 71 and the second portion 72 may be formed of a material having a Young's modulus in a range from 193 to 206 GPa. The Young's modulus of the material constituting the first portion 71 may be different from the Young's modulus of the material constituting the second portion 72. For example, the material constituting the first portion 71 may be harder than the material constituting the second portion 72. In the gas sensor 1, the ceramic housing 60 is formed of ceramics, whereas the hollow member 70 and the main metal fitting 21 are formed of metal, for example. Therefore, among the main metal fitting 21, the hollow member 70, and the ceramic housing 60, it is considered that the ceramic housing 60 is most likely to be damaged (for example, a chip) by an impact applied to the gas sensor 1. Therefore, in the hollow member 70, the “portion on the rear end side (second portion 72) in contact with the ceramic housing 60” may be softer than the “portion on the distal end side (first portion 71) in contact with the main metal fitting 21”. In addition, the Young's modulus of the material constituting the first portion 71 may be the same as the Young's modulus of the material constituting the second portion 72, and for example, the material constituting the first portion 71 and the material constituting the second portion 72 may be the same. The hollow member 70 may be formed of a material having a Young's modulus in a range from 193 to 206 GPa. For example, the entire hollow member 70 may be formed of a material having a Young's modulus in a range from 193 to 206 GPa.

The hollow member 70 may be formed of a thin plate having a plate thickness in a range from 0.3 to 0.8 mm, for example, may be formed of a metal plate having a plate thickness in a range from 0.3 to 0.8 mm.

Here, a heat source exists on the distal end side of the gas sensor 1, so that heat generated by the heat source may be transferred to the ceramic housing 60 via the hollow member 70 and further to the elastic body 50 via the ceramic housing 60. When heat is transferred to the ceramic housing 60, for example, the contact between the connector electrode 12 of the sensor element 10 and the element contact portion 31 of the terminal metal fitting 30 is heated to a high temperature, whereby the contact is oxidized and the contact resistance may increase. In addition, when heat is transferred to the elastic body 50, the elastic body 50 may be eroded. When erosion occurs in the elastic body 50, the reference gas (reference air) in the outer tube 22 may be contaminated or the seismic resistance of the gas sensor 1 may deteriorate. In addition, an electrode (for example, a reference electrode provided so as to be contactable with a reference gas) included in the sensor element 10 may be contaminated by an organic gas accompanying decomposition of the elastic body 50 by heat.

Therefore, the present inventors have studied suppressing heat transfer to the ceramic housing 60 and the like while maintaining the strength of the hollow member 70 that prevents the ceramic housing 60 from moving to the distal end side in the axial direction by devising the configuration of the hollow member 70. The present inventors have conducted an experiment (second test described later) and confirmed that the hollow member 70 formed of a thin plate having a plate thickness in a range from 0.3 to 0.8 mm can remarkably suppress heat transfer to the ceramic housing 60 and the like while maintaining the strength of the hollow member 70. The present inventors have confirmed that the thin plate having a plate thickness in a range from 0.3 to 0.8 mm has sufficient strength to prevent the ceramic housing 60 and the like from moving to the distal end side in the axial direction and has sufficient heat capacity to suppress heat transfer to the ceramic housing 60.

Therefore, the hollow member 70 formed of a thin plate having a plate thickness in a range from 0.3 to 0.8 mm can remarkably suppress heat transfer to the ceramic housing 60 and the like while having sufficient strength to prevent the ceramic housing 60 from moving to the distal end side in the axial direction. Therefore, the gas sensor 1 can prevent the ceramic housing 60 from moving to the distal end side in the axial direction while suppressing a risk of breakage of a member constituting the gas sensor 1, and can further improve heat resistance of the gas sensor 1.

As described above, in the gas sensor 1, the hollow member 70 includes the first portion 71 and the second portion 72, and the material (member) constituting the first portion 71 and the material (member) constituting the second portion 72 may be different from each other. That is, the hollow member 70 formed of a material having a Young's modulus in a range from 193 to 206 GPa may be constituted of a plurality of members. For example, the first portion 71 and the second portion 72 included in the hollow member 70 may have a Young's modulus in a range from 193 to 206 GPa and may be formed of materials different from each other. The hollow member 70 may be formed of a plurality of members (for example, a member constituting the first portion 71 and a member constituting the second portion 72), and each of the plurality of members may be formed of a material having a Young's modulus in a range from 193 to 206 GPa. In this case, in the gas sensor 1, the hollow member 70 formed of a material having a Young's modulus in a range from 193 to 206 GPa can be easily made of the plurality of members.

In the gas sensor 1, the hollow member 70 may further include a member other than the member corresponding to the first portion 71 and the member corresponding to the second portion 72, and may further include, for example, a third cylindrical member and a fourth cylindrical member. For the gas sensor 1, the hollow member 70 is not necessarily composed of two members (a member corresponding to the first portion 71 and a member corresponding to the second portion 72), and the hollow member 70 may be composed of three or more members. When the hollow member 70 is formed of a plurality of members, each of the plurality of members may be formed of a material having a Young's modulus in a range from 193 to 206 GPa. However, the hollow member 70 may be formed of a material having a Young's modulus in a range from 193 to 206 GPa, and it is not essential that the hollow member 70 be made of a plurality of members. For example, the member corresponding to the first portion 71 and the member corresponding to the second portion 72 may be integrally configured, and the hollow member 70 may include one member. The entire hollow member 70 may be formed of a material having a Young's modulus in a range from 193 to 206 GPa. Similarly to the Young's modulus, the first portion 71 and the second portion 72 may have different plate thicknesses. That is, the plate thickness of the thin plate constituting the first portion 71 may be different from the plate thickness of the thin plate constituting the second portion 72, and each of the first portion 71 and the second portion 72 may be formed of a thin plate having a plate thickness in a range from 0.3 to 0.8 mm. Further, the plate thickness of the thin plate constituting the first portion 71 may be the same as the plate thickness of the thin plate constituting the second portion 72, and for example, the thin plate constituting the first portion 71 and the thin plate constituting the second portion 72 may be the same. When the hollow member 70 is formed of a plurality of members, each of the plurality of members may be formed of a thin plate having a plate thickness in a range from 0.3 to 0.8 mm. However, the hollow member 70 only needs to be formed of a thin plate having a plate thickness in a range from 0.3 to 0.8 mm, and it is not essential that the hollow member 70 be made of a plurality of members. For example, the entire hollow member 70 may be formed of a thin plate having a plate thickness in a range from 0.3 to 0.8 mm.

Although the main metal fitting 21 and the hollow member 70 in contact with the rear end of the main metal fitting 21 are illustrated as separate members in FIG. 1, the main metal fitting 21 and the hollow member 70 may be integrally formed in the gas sensor 1. For example, a member integrally configured to have a hollow inside with an open end of a thick cylindrical member (first cylinder) and an open end of a thin cylindrical member (second cylinder) facing each other may be a “member in which the main metal fitting 21 and the hollow member 70 are integrally configured”. The first cylinder fixes the sensor element 10 penetrating the inside in the axial direction via the annular component 90 (holding member), and corresponds to the main metal fitting 21. The second cylinder is disposed coaxially with the first cylinder on the rear end side of the first cylinder, is in contact with the distal end side of the ceramic housing 60 on the rear end side in a state where the sensor element 10 penetrates the inside in the axial direction, and corresponds to the hollow member 70. The second cylinder is formed of a material having a Young's modulus in a range from 193 to 206 GPa. Further, the second cylinder may be formed of a thin plate having a plate thickness in a range from 0.3 to 0.8 mm. By integrally forming the main metal fitting 21 and the hollow member 70, the gas sensor 1 has the following effects. That is, the gas sensor 1 has an effect that the hollow member 70 “preventing the ceramic housing 60 from moving to the distal end side in the axial direction while suppressing the risk of damaging members constituting the gas sensor 1” can be easily configured together with the main metal fitting 21. The gas sensor 1 has also an effect of suppressing the number of members constituting the gas sensor 1.

In FIG. 1, the sensor element 10 penetrates the inside of the hollow member 70 in the axial direction, and in particular, the sensor element 10 penetrates the inside of the hollow member 70 in the axial direction without contacting the hollow member 70. That is, in the gas sensor 1, the hollow member 70 is spaced apart from the sensor element 10, that is, the hollow member 70 and the sensor element 10 are not in contact with each other. Therefore, the gas sensor 1 has an effect of reducing a risk that the hollow member 70 and the sensor element 10 interfere with each other to cause element breakage or the like. For example, even when the hollow member 70 is inclined from the axial direction and the like due to application of an external force to the gas sensor 1 and the like, the hollow member 70 is spaced apart from the sensor element 10 and thus does not interfere with the sensor element 10.

For example, the hollow member 70 may be configured such that the inner peripheral surface of the hollow member 70 and the sensor element 10 are spaced apart from each other by a predetermined distance or more in the radial direction. Here, as described above, the sensor element 10 is an elongated flat plate-like (elongated plate-like) element. Therefore, for example, the hollow member 70 may be configured such that a distance between the inner peripheral surface of the hollow member 70 and the sensor element 10 in at least the thickness direction of the sensor element 10 is equal to or larger than the thickness of the sensor element 10. As an example, the hollow member 70 may be configured such that the distance between the inner peripheral surface of the hollow member and the sensor element 10 in the thickness direction of the sensor element 10 is twice or more the thickness of the sensor element 10. By configuring the hollow member 70 so that the inner peripheral surface and the sensor element 10 are sufficiently spaced apart from each other in the radial direction (for example, in the thickness direction of the sensor element 10), the gas sensor 1 can more reliably prevent interference between the hollow member 70 and the sensor element 10.

### <Details of Arrangement Situation of Hollow Member>

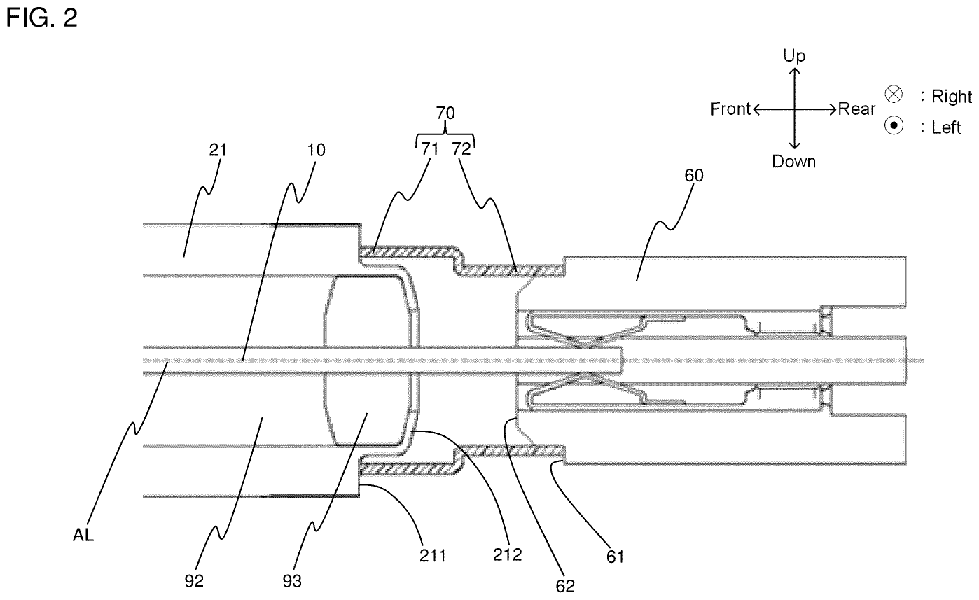

FIG. 2 is an enlarged cross-sectional view schematically illustrating a main part of the gas sensor 1, specifically, an enlarged cross-sectional view of a main part illustrating an arrangement state of the hollow member 70 of the gas sensor 1 in an enlarged manner. In FIG. 2, the horizontal direction in the drawing is the axial direction (longitudinal direction) of the gas sensor 1 (sensor element 10), and the left side in the drawing is the distal end side and the right side in the drawing is the rear end side.

As illustrated in FIG. 2, the main metal fitting 21 includes, for example, a cylindrical base portion in which the annular component 90 is enclosed inside, and a cylindrical swaging portion 212 that is provided on the rear end side of the base portion and presses the position on the rear end side of the annular component 90 in a bent state.

In FIG. 2, only green compact 92 and second ceramic supporter 93 of first ceramic supporter 91, green compact 92, and second ceramic supporter 93, which constitute annular component 90, are illustrated.

The swaging portion 212 is formed over the entire circumference in the circumferential direction by performing swaging processing on, for example, the “cylinder formed of a metal plate having a thickness smaller than that of the base portion, the metal plate further extending from a rear end portion of the base portion toward a rear end side”, and is bent inward in the radial direction. The swaging portion 212 fixes the annular component 90 to the main metal fitting 21. The thickness of the swaging portion 212 is thinner than the thickness of the base portion.

As described above, in the main metal fitting 21, the swaging portion 212 is formed on the rear end side of the base portion, and the thickness of the base portion is thicker than the thickness of the swaging portion 212. Therefore, the base portion has a rear end surface 211 configured as a difference between the thickness of the base portion and the thickness of the swaging portion 212 on the rear end side thereof, and the rear end surface 211 is inclined with respect to the axial direction. In the example illustrated in FIG. 2, the rear end surface 211 is orthogonal to the axial direction, that is, parallel to the radial direction.

Then, the hollow member 70 is in contact with the rear end side of the main metal fitting 21 at the distal end side thereof, and in the example shown in FIG. 2, the first portion 71 of the hollow member 70 is in contact with the rear end surface 211 of the main metal fitting 21 (base portion). That is, in the gas sensor 1, the main metal fitting 21 includes the rear end surface 211 orthogonal to the axial direction on the rear end side, and the hollow member 70 (in particular, the first portion 71) is in contact with the rear end surface 211 of the main metal fitting 21.

Therefore, the gas sensor 1 can easily and stably position the hollow member 70 in the axial direction using the surface (rear end surface 211) orthogonal to the axial direction of the main metal fitting 21, and particularly, can easily and stably restrict the movement of the hollow member 70 toward the distal end side. The gas sensor 1 can also easily and stably position the hollow member 70 in the axial direction to easily and stably position the ceramic housing 60 restricted in movement to the distal end side by the hollow member 70 in the axial direction.

However, for the gas sensor 1, it is not essential that the rear end surface 211 of the main metal fitting 21 (base portion) be orthogonal to the axial direction, that is, it is not essential that the rear end surface 211 extend in the radial direction. Further, it is not essential that the hollow member 70 (in particular, the first portion 71) be in contact with the rear end surface 211 of the main metal fitting 21 (base portion). In the gas sensor 1, the hollow member 70 only needs to be in contact with the rear end side of the main metal fitting 21, and may be in contact with, for example, the swaging portion 212.

As described with reference to FIG. 1, the hollow member 70 is in contact with the main metal fitting 21 (in particular, the rear end side of the main metal fitting 21) on the distal end side in the axial direction, and is in contact with the ceramic housing 60 (in particular, the distal end side of the ceramic housing 60) on the rear end side in the axial direction. Since the distal end side (that is, the first portion 71) of the hollow member 70 in the axial direction is in contact with the main metal fitting 21, the movement of the hollow member in the axial direction is restricted at least to the distal end side. For example, the hollow member 70 may be fixed to the main metal fitting 21 to restrict the movement in the axial direction at least to the distal end side, and in one example, may be welded to the main metal fitting 21 to restrict the movement in the axial direction (in particular, movement to the distal end side). The hollow member 70, which is fixed to the main metal fitting 21 to restrict the movement of the hollow member to the distal end side, may be in contact with the ceramic housing 60 on the rear end side (that is, the second portion 72) to prevent the movement of the ceramic housing 60 to the distal end side.

In addition, as exemplified in FIG. 2, the ceramic housing 60 includes one or more (for example, a plurality of) surfaces inclined with respect to the axial direction (for example, the axial direction is orthogonal to the axial direction) in the end surface on the distal end side in the axial direction. For example, the ceramic housing 60 shown in FIG. 2 includes a first distal end surface 61 and a second distal end surface 62 on the distal end side in the axial direction, and the first distal end surface 61 and the second distal end surface 62 are each inclined with respect to the axial direction. In particular, in the example illustrated in FIG. 2, each of the first distal end surface 61 and the second distal end surface 62 is orthogonal to the axial direction, that is, parallel to the radial direction.

The hollow member 70 is in contact with the distal end side of the ceramic housing 60 on the rear end side thereof, and in the example shown in FIG. 2, the second portion 72 of the hollow member 70 is in contact with the first distal end surface 61 of the ceramic housing 60. That is, in the gas sensor 1, the ceramic housing 60 includes the first distal end surface 61 orthogonal to the axial direction on the distal end side, and the hollow member 70 (in particular, the second portion 72) is in contact with the first distal end surface 61 of the ceramic housing 60.

Here, as described above, in the gas sensor 1, the distal end side (first portion 71) of the hollow member 70 is in contact with the main metal fitting 21, and the main metal fitting 21 restricts the movement of the hollow member toward the distal end side. The ceramic housing 60 is in contact with the “hollow member 70 restricted in movement to the distal end side”, and in particular, the ceramic housing 60 is in contact with the rear end side (second portion 72) of the hollow member 70 at the first distal end surface 61 orthogonal to the axial direction. Therefore, the gas sensor 1 can easily and stably implement the positioning of the ceramic housing 60 in the axial direction using the first distal end surface 61, and particularly, can easily and stably restrict the movement of the ceramic housing 60 to the distal end side. In the gas sensor 1, the first distal end surface 61 of the ceramic housing 60 orthogonal to the axial direction is in contact with the rear end side (second portion 72) of the hollow member 70 restricted in movement to the distal end side, so that movement to the distal end side of the ceramic housing 60 can be easily and stably restricted.

The rear end side (second portion 72) of the hollow member 70 only needs to be in contact with the ceramic housing 60, and for example, only needs to be in contact with a “surface inclined with respect to the axial direction” on the distal end side in the axial direction of the ceramic housing 60. It is not essential for the gas sensor 1 that the “surface of ceramic housing 60 on distal end side in axial direction” with which the rear end side of the hollow member 70 is in contact is orthogonal to the axial direction. In addition, it is not essential for the ceramic housing 60 to include a plurality of surfaces inclined with respect to the axial direction on the distal end side in the axial direction. The “surface inclined with respect to the axial direction” provided on the distal end side in the axial direction of the ceramic housing 60 may be one.

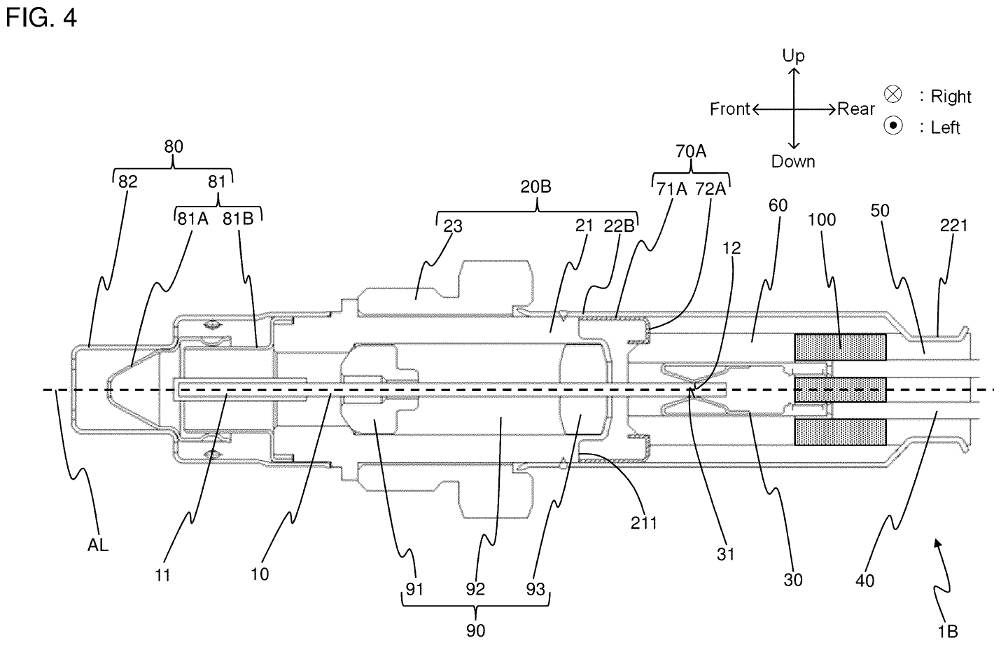

In the gas sensor exemplified in FIG. 1, the ceramic housing 60 is restricted from moving to the rear end side in the axial direction by the elastic body 50. That is, in FIG. 1, the rear end side of the ceramic housing 60 is in contact with the distal end side (specifically, the end surface on the distal end side) of the elastic body 50, and the elastic body 50 restricts the movement to the rear end side. However, in the gas sensor according to one aspect of the present invention, it is not essential that the movement of the ceramic housing 60 to the rear end side is restricted by the elastic body 50, and it is not essential that the ceramic housing 60 is in contact with the elastic body 50. In the gas sensor according to an aspect of the present invention, the movement of the ceramic housing 60 to the distal end side may be restricted by a hollow member (for example, the hollow member 70), and the movement of the ceramic housing 60 to the rear end side may not be restricted by the elastic body 50. The movement of the ceramic housing 60 to the rear end side may be restricted by, for example, an outer tube, or the movement to the rear end side may be restricted by a member (for example, spacer 100 of FIG. 4) disposed between the ceramic housing 60 and the elastic body 50. Details of these examples will be described later.

### <Consideration on Shape of Hollow Member>

So far, with reference to FIGS. 1 and 2, the hollow member 70 whose outer diameter changes in the axial direction has been described as a hollow member for preventing the ceramic housing 60 from moving to the distal end side in the axial direction while suppressing the risk of breakage of the member constituting the gas sensor. That is, the hollow member 70 described so far includes the first portion 71 in contact with the rear end side of the main metal fitting 21 and the second portion 72 in contact with the distal end side of the ceramic housing 60, and the outer diameter of the first portion 71 and the outer diameter of the second portion 72 are different. However, it is not essential for the hollow member included in the gas sensor according to one aspect of the present invention that the outer diameter changes in the axial direction. Hereinafter, a hollow member 70A having a constant outer diameter in the axial direction (that is, the outer diameter does not change) will be described as a hollow member for preventing the ceramic housing 60 from moving to the distal end side in the axial direction while suppressing the risk of damaging a member constituting the gas sensor.

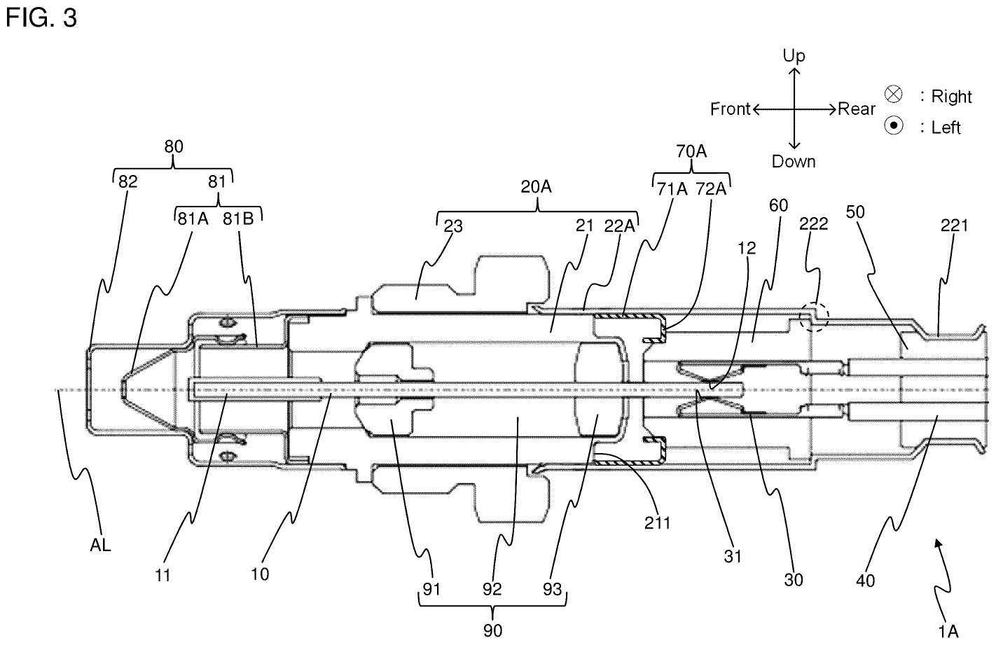

FIG. 3 is a schematic cross-sectional view schematically illustrating an example of a main configuration of a gas sensor 1A according to a first modification. In FIG. 3, the horizontal direction on the sheet surface is the axial direction (longitudinal direction) of the gas sensor 1A, the left side on the sheet surface is the distal end side, and the right side on the sheet surface is the rear end side. The gas sensor 1A illustrated in FIG. 3 includes a hollow member 70A in order to prevent the ceramic housing 60 from moving to the distal end side in the axial direction while suppressing the risk of damaging the members constituting the gas sensor 1A. The gas sensor 1A illustrated in FIG. 3 is similar to the gas sensor 1 described with reference to FIGS. 1 and 2 except for the following two points. First, the gas sensor 1A includes a hollow member 70A instead of the hollow member 70. Secondly, in the gas sensor 1A, the movement of the ceramic housing 60 to the rear end side is restricted by the outer tube 22A instead of the elastic body 50. Since the gas sensor 1A is similar to the gas sensor 1 except for the above two points, detailed description of configurations other than the hollow member 70A and the outer tube 22A will be omitted in the following description.

### (Hollow Member)

The hollow member 70A is a hollow member having a cylindrical (columnar) appearance, and in particular, is a hollow member having a cylindrical appearance whose outer diameter is constant in the axial direction. The hollow member 70A illustrated in FIG. 3 is configured by, for example, folding back one end (rear end in the illustrated example) of a cylindrical member radially inward over the entire circumferential direction. The hollow member 70A illustrated in FIG. 3 is a hollow member in which each of two rear end portions is configured in a substantially U shape in a cross section parallel to the axis AL and in contact with the axis AL, and an outer diameter of the hollow member 70A is constant in the axial direction. That is, the hollow member 70A includes a first portion 71A arranged on the distal end side in the axial direction, and a second portion 72A arranged on the rear end side of the first portion 71A in the axial direction and having the same outer diameter as the first portion 71A. The first portion 71A is a cylindrical portion provided on the distal end side of the hollow member 70A, and is in contact with the rear end side of the main metal fitting 21, and in the example illustrated in FIG. 3, is in contact with the rear end surface 211 provided on the rear end side of the main metal fitting 21. The second portion 72A is a portion on the rear end side of the hollow member 70A, specifically, a portion extending from the first portion 71A to the rear end side. Each of the two rear ends of the second portion 72A is formed in a substantially U shape in a cross section parallel to the axis AL and in contact with the axis AL. The second portion 72A is in contact with the ceramic housing 60 at the rear end thereof, particularly, the distal end side of the ceramic housing 60. The second portion 72A is configured by, for example, folding back a rear end side of a “cylindrical member further extending from a rear end portion of the first portion 71A to a rear end side” radially inward over the entire circumferential direction. In addition, the first portion 71A and the second portion 72A have a cylindrical (columnar) appearance with outer diameters equal to each other. However, for the hollow member according to one aspect of the present invention, it is not essential that the outer diameter of the first portion 71A and the outer diameter of the second portion 72A are equal, and the outer diameters of the first portion 71A and the second portion 72A may be different from each other.

In the gas sensor 1A, the hollow member 70A is disposed between the main metal fitting 21 and the ceramic housing 60 in the axial direction. Specifically, the hollow member 70A is disposed inside the outer tube 22A in a state where a distal end side (that is, the first portion 71A) is in contact with a rear end side of the main metal fitting 21 and a rear end side (that is, the second portion 72A) is in contact with a distal end side of the ceramic housing 60. The sensor element 10 penetrates the inside of the hollow member 70A in the axial direction, that is, the sensor element 10 penetrates the inside of the first portion 71A and the second portion 72A in the axial direction.

That is, in the gas sensor 1A, the hollow member 70A is in contact with the rear end side of the main metal fitting 21 on the distal end side in the axial direction, so that the movement of the hollow member 70A to the distal end side is restricted by the main metal fitting 21. Then, the ceramic housing 60 is in contact with the hollow member 70A on the distal end side thereof, that is, the hollow member 70A restricted in movement to the distal end side. Therefore, in the gas sensor 1A, the movement of the ceramic housing 60 to the distal end side is restricted by the hollow member 70A, that is, the hollow member 70A prevents the ceramic housing 60 from moving to the distal end side.

The hollow member 70A is formed of a material having a Young's modulus in a range from 193 to 206 GPa, for example, a metal plate having a Young's modulus in a range from 193 to 206 GPa. Therefore, the hollow member 70A formed of a material having a Young's modulus in a range from 193 to 206 GPa can remarkably suppress the occurrence of breakage or the like in the ceramic housing 60, particularly in a contact portion of the ceramic housing 60 with respect to the hollow member 70A. That is, the hollow member 70A formed of a material having a Young's modulus in a range from 193 to 206 GPa can remarkably suppress the occurrence of damage and the like at the contact portion of the ceramic housing 60 with respect to the hollow member 70A. In the hollow member 70A, due to the contact portion (second portion 72A) with respect to the ceramic housing 60 being sufficiently soft, it is possible to prevent, for example, the occurrence of a situation in which “the ceramic housing 60 is damaged by collision with the hollow member 70A”. In addition, the hollow member 70A formed of a material having a Young's modulus in a range from 193 to 206 GPa has sufficient hardness that can reliably (stably) prevent movement of the ceramic housing 60 toward the distal end side. That is, since the hollow member 70A is formed of a material having a Young's modulus in a range from 193 to 206 GPa, it is possible to reliably (stably) prevent the movement of the hollow member toward the distal end side of the ceramic housing 60 without being deformed or damaged. As described above, in the gas sensor 1A, the hollow member 70A can prevent the ceramic housing 60 from moving to the distal end side in the axial direction while suppressing the risk of damaging the member (for example, the ceramic housing 60) constituting the gas sensor 1A.

In the gas sensor 1A, (1) hermetic sealing between the distal end side and the rear end side of the sensor element is realized by the main metal fitting 21 (and the annular component 90), and (2) restriction of movement to the distal end side of the ceramic housing 60 is realized by the hollow member 70A. Therefore, the gas sensor 1A can achieve both the hermetic sealing and the movement restriction described above without causing a problem that a conventional gas sensor has, such as “in order to realize the above-mentioned hermetic sealing, it is difficult to realize the movement restriction”.

As described above, the hollow member 70A is in contact with the main metal fitting 21 (in particular, the rear end side of the main metal fitting 21) on the distal end side in the axial direction, and is in contact with the ceramic housing 60 (in particular, the distal end side of the ceramic housing 60) on the rear end side in the axial direction. Since the distal end side (that is, the first portion 71A) of the hollow member 70A in the axial direction is in contact with the main metal fitting 21, the movement of the hollow member in the axial direction is restricted at least to the distal end side. For example, the hollow member 70A may be fixed to the main metal fitting 21 to restrict the movement in the axial direction at least to the distal end side, and in one example, the hollow member may be welded to the main metal fitting 21 to restrict the movement in the axial direction (in particular, movement to the distal end side). The hollow member 70A, which is fixed to the main metal fitting 21 to restrict the movement of the hollow member to the distal end side, may be in contact with the ceramic housing 60 on the rear end side (that is, the second portion 72A) to prevent the movement of the ceramic housing 60 to the distal end side.

Similarly to the hollow member 70, the hollow member 70A may be formed of a thin plate having a plate thickness in a range from 0.3 to 0.8 mm, for example, may be formed of a metal plate having a plate thickness in a range from 0.3 to 0.8 mm. The hollow member 70A formed of a thin plate having a plate thickness in a range from 0.3 to 0.8 mm can remarkably suppress heat transfer to the ceramic housing 60 and the like while having sufficient strength to prevent the ceramic housing 60 from moving to the distal end side in the axial direction. Therefore, the gas sensor 1A can prevent the ceramic housing 60 from moving to the distal end side in the axial direction while suppressing a risk of breakage of a member constituting the gas sensor 1A, and can further improve heat resistance of the gas sensor 1A.

In the gas sensor 1A, the material (member) constituting the first portion 71A and the material (member) constituting the second portion 72A may be different from each other. In the hollow member 70A, each of the first portion 71A and the second portion 72A may be formed of a material having a Young's modulus in a range from 193 to 206 GPa. The Young's modulus of the material constituting the first portion 71A may be different from the Young's modulus of the material constituting the second portion 72A. For example, the material constituting the first portion 71A may be harder than the material constituting the second portion 72A. That is, the hollow member 70A formed of a material having a Young's modulus in a range from 193 to 206 GPa may be constituted of a plurality of members. For example, the first portion 71A and the second portion 72A included in the hollow member 70A may have a Young's modulus in a range from 193 to 206 GPa and may be formed of materials different from each other. The hollow member 70A may include a plurality of members (for example, a member constituting the first portion 71A and a member constituting the second portion 72A) each including a material having a Young's modulus in a range from 193 to 206 GPa. In this case, in the gas sensor 1A, the hollow member 70A formed of a material having a Young's modulus in a range from 193 to 206 GPa can be easily made of the plurality of members. In the gas sensor 1A, the hollow member 70A formed of a material having a Young's modulus in a range from 193 to 206 GPa can be easily formed of, for example, a plurality of members including a member corresponding to the first portion 71A and a member corresponding to the second portion 72A.

In the gas sensor 1A, the hollow member 70A may further include a member other than the member corresponding to the first portion 71A and the member corresponding to the second portion 72A in addition to these members. The hollow member 70A may further include, for example, a third cylindrical member, a fourth cylindrical member, and the like in addition to the member corresponding to the first portion 71A and the member corresponding to the second portion 72A. For the gas sensor 1A, the hollow member 70A is not necessarily composed of two members (member corresponding to first portion 71A and member corresponding to second portion 72A), and the hollow member 70A may be composed of three or more members. When the hollow member 70A is formed of a plurality of members, each of the plurality of members may be formed of a material having a Young's modulus in a range from 193 to 206 GPa. However, the hollow member 70A may be formed of a material having a Young's modulus in a range from 193 to 206 GPa, and it is not essential that the hollow member 70A be made of a plurality of members. For example, the member corresponding to the first portion 71A and the member corresponding to the second portion 72A may be integrally configured, and the hollow member 70A may include one member. The entire hollow member 70A may be formed of a material having a Young's modulus in a range from 193 to 206 GPa.

Similarly to the Young's modulus, the first portion 71A and the second portion 72A may have different plate thicknesses. That is, the plate thickness of the thin plate constituting the first portion 71A may be different from the plate thickness of the thin plate constituting the second portion 72A, and each of the first portion 71A and the second portion 72A may be formed of a thin plate having a plate thickness in a range from 0.3 to 0.8 mm. Further, the plate thickness of the thin plate constituting the first portion 71A may be the same as the plate thickness of the thin plate constituting the second portion 72A, and for example, the thin plate constituting the first portion 71A and the thin plate constituting the second portion 72A may be the same. When the hollow member 70A is formed of a plurality of members, each of the plurality of members may be formed of a thin plate having a plate thickness in a range from 0.3 to 0.8 mm. However, the hollow member 70A only needs to be formed of a thin plate having a plate thickness in a range from 0.3 to 0.8 mm, and it is not essential that the hollow member 70A be made of a plurality of members. For example, the entire hollow member 70A may be formed of a thin plate having a plate thickness in a range from 0.3 to 0.8 mm.

Although the main metal fitting 21 and the hollow member 70A in contact with the rear end of the main metal fitting 21 are illustrated as separate members in FIG. 3, the main metal fitting 21 and the hollow member 70A may be integrally formed in the gas sensor 1A. By integrally forming the main metal fitting 21 and the hollow member 70A, the gas sensor 1A has the following effects. That is, the gas sensor 1A has an effect that the hollow member 70A “preventing the ceramic housing 60 from moving to the distal end side in the axial direction while suppressing the risk of damaging members constituting the gas sensor 1A” can be easily configured together with the main metal fitting 21. The gas sensor 1A has also an effect of suppressing the number of members constituting the gas sensor 1A.

Similarly to the main metal fitting 21 in the gas sensor 1, the main metal fitting 21 in the gas sensor 1A includes the rear end surface 211 inclined with respect to the axial direction on the rear end side, and includes, for example, the rear end surface 211 orthogonal to the axial direction. Then, the hollow member 70A is in contact with the rear end side of the main metal fitting 21 at the distal end side thereof, and in the example shown in FIG. 3, the first portion 71A of the hollow member 70A is in contact with the rear end surface 211 of the main metal fitting 21 (in particular, the base portion of the main metal fitting 21).

Therefore, the gas sensor 1A can easily and stably position the hollow member 70A in the axial direction using the surface (rear end surface 211) orthogonal to the axial direction of the main metal fitting 21, and particularly, can easily and stably restrict the movement of the hollow member 70A toward the distal end side. The gas sensor 1A can also easily and stably position the hollow member 70A in the axial direction to easily and stably position the ceramic housing 60 restricted in movement to the distal end side by the hollow member 70A in the axial direction.

In addition, as described above, the ceramic housing 60 includes the first distal end surface 61 and the second distal end surface 62 on the distal end side in the axial direction, and the first distal end surface 61 and the second distal end surface 62 are each inclined with respect to the axial direction, and are, for example, orthogonal to the axial direction. The hollow member 70A is in contact with the distal end side of the ceramic housing 60 on the rear end side thereof, and in the example shown in FIG. 3, the second portion 72A of the hollow member 70A is in contact with the first distal end surface 61 of the ceramic housing 60. Further, a distal end side (first portion 71A) of the hollow member 70A is in contact with the main metal fitting 21, and movement of the hollow member toward the distal end side is restricted by the main metal fitting 21. Therefore, the gas sensor 1A can easily and stably implement the positioning of the ceramic housing 60 in the axial direction using the first distal end surface 61, and particularly, can easily and stably restrict the movement of the ceramic housing 60 to the distal end side. In the gas sensor 1A, the first distal end surface 61 of the ceramic housing 60 orthogonal to the axial direction is in contact with the rear end side (second portion 72A) of the hollow member 70A restricted in movement to the distal end side, so that movement to the distal end side of the ceramic housing 60 can be easily and stably restricted.

In FIG. 3, the sensor element 10 penetrates the inside of the hollow member 70A in the axial direction, and in particular, the sensor element 10 penetrates the inside of the hollow member 70A in the axial direction without contacting the hollow member 70A. That is, in the gas sensor 1A, the hollow member 70A is spaced apart from the sensor element 10, that is, the hollow member 70A and the sensor element 10 are not in contact with each other. Therefore, the gas sensor 1A has an effect of reducing a risk that the hollow member 70A and the sensor element 10 interfere with each other to cause element breakage or the like. For example, even when the hollow member 70A is inclined from the axial direction and the like due to application of an external force to the gas sensor 1A and the like, the hollow member 70A is spaced apart from the sensor element 10 and thus does not interfere with the sensor element 10.

For example, the hollow member 70A may be configured such that the inner peripheral surface of the hollow member 70A and the sensor element 10 are spaced apart from each other by a predetermined distance or more in the radial direction. For example, the hollow member 70A may be configured such that a distance between the inner peripheral surface of the hollow member 70A and the sensor element 10 in at least the thickness direction of the sensor element 10 is equal to or larger than the thickness of the sensor element 10. As an example, the hollow member 70A may be configured such that the distance between the inner peripheral surface of the hollow member and the sensor element 10 in the thickness direction of the sensor element 10 is twice or more the thickness of the sensor element 10. By configuring the hollow member 70A so that the inner peripheral surface and the sensor element 10 are sufficiently spaced apart from each other in the radial direction (for example, in the thickness direction of the sensor element 10), the gas sensor 1A can more reliably prevent interference between the hollow member 70A and the sensor element 10.

### (Outer Tube)

As described above, in the gas sensor 1A, the movement of the ceramic housing 60 to the rear end side is restricted by the outer tube 22A instead of the elastic body 50. Therefore, in the gas sensor 1A, the ceramic housing 60 may not be in contact with the elastic body 50. In the gas sensor 1A exemplified in FIG. 3, the ceramic housing 60 is spaced apart from the elastic body 50, that is, the ceramic housing 60 and the elastic body 50 are not in contact with each other. By separating the elastic body 50 from the ceramic housing 60 disposed on the distal end side of the elastic body 50 in the axial direction, the gas sensor 1A has the following effects. That is, the gas sensor 1A can prevent heat generated by the heat source on the distal end side of the gas sensor 1A from being transferred to the elastic body 50 via the ceramic housing 60, and can prevent a situation such as erosion of the elastic body 50 caused by the heat.

In the gas sensor 1A, the inner peripheral surface of the outer tube 22A accommodating the ceramic housing 60 therein includes a portion (locking portion 222) inclined from the axial direction on the rear end side, and in the example shown in FIG. 3, the locking portion 222 is orthogonal (substantially orthogonal) to the axial direction. The outer tube 22A exemplified in FIG. 3 includes a portion having a smaller diameter (reduced diameter portion) on the rear end side (in particular, on the rear end side on the distal end side with respect to the end surface on the distal end side of the elastic body 50). In the reduced diameter portion, the inner peripheral surface of the outer tube 22A is inclined with respect to the axial direction. In the illustrated example, in the reduced diameter portion, the inner peripheral surface of the outer tube 22A is orthogonal (substantially orthogonal) to the axial direction. That is, the inner peripheral surface of the outer tube 22A is inclined with respect to the axial direction in the reduced diameter portion, and a portion inclined with respect to the axial direction is the locking portion 222. In the locking portion 222, the ceramic housing 60 is in contact with the inner peripheral surface of the outer tube 22A, that is, the ceramic housing 60 is in contact with the locking portion 222 on the inner peripheral surface of the outer tube 22A. The ceramic housing 60 is restricted from moving to the rear end side in the axial direction by being in contact with the locking portion 222 on the inner peripheral surface of the outer tube 22A on the rear end side in the axial direction, that is, the outer tube 22A restricts the movement of the ceramic housing 60 to the rear end side. Therefore, the gas sensor 1A can prevent the ceramic housing 60 from moving to the rear end side in the axial direction without bringing the ceramic housing 60 into contact with the elastic body 50.

In particular, as described above, the inner peripheral surface of the outer tube 22A in the locking portion 222 is inclined from the axial direction, and is, for example, orthogonal (substantially orthogonal) to the axial direction. Therefore, the gas sensor 1A can easily and stably realize the positioning of the ceramic housing 60 in the axial direction using the locking portion 222, and particularly, can easily and stably restrict the movement of the ceramic housing 60 to the rear end side. For example, in the gas sensor 1A, the end surface (for example, an end surface on the rear end side orthogonal to the axial direction) on the rear end side of the ceramic housing 60 is in contact with the locking portion 222 orthogonal to the axial direction, so that the movement of the ceramic housing 60 to the rear end side can be easily and stably restricted.

However, it is not essential that the inner peripheral surface of the outer tube 22A is orthogonal (substantially orthogonal) to the axial direction in the locking portion 222, and it is sufficient that the inner peripheral surface of the outer tube 22A is inclined with respect to the axial direction in the locking portion 222. In addition, it is not essential that the end surface on the rear end side of the ceramic housing 60 (that is, the end surface on the rear end side of the ceramic housing 60, which is in contact with the inner peripheral surface of the outer tube 22A at the locking portion 222) be orthogonal to the axial direction, and the end surface on the rear end side may be inclined with respect to the axial direction.

The outer tube 22A is similar to the outer tube 22 except that (1) the above-described reduced diameter portion is formed on the rear end side (in particular, on the rear end side on the distal end side with respect to the end surface on the distal end side of the elastic body 50), and (2) the hollow member 70A is accommodated instead of the hollow member 70. For example, the outer tube 22A is a tubular member extending in the axial direction, and is, for example, a metal cylindrical member. The outer tube 22A is attached to a portion on the rear end side of the main metal fitting 21, accommodates the rear end of the sensor element 10, the hollow member 70A, and the ceramic housing 60 (terminal metal fitting 30) therein, and has an open end on the rear end side sealed with an elastic body 50.

### <Consideration on Configuration for Restricting Movement of Ceramic Housing to Rear End Side>

The gas sensor 1 that restricts the movement of the ceramic housing 60 to the rear end side by the elastic body 50 and the gas sensor 1A that restricts the movement of the ceramic housing 60 to the rear end side by the outer tube 22A have been described. However, in the gas sensor according to an aspect of the present invention, the movement of the ceramic housing 60 to the distal end side may be restricted by a hollow member (for example, the hollow member 70, 70A), and a method of restricting the movement of the ceramic housing 60 to the rear end side is not particularly limited. Hereinafter, the gas sensor 1B that restricts the movement of the ceramic housing 60 toward the rear end side by a member that is neither the elastic body 50 nor the outer tube 22A will be described with reference to FIG. 4.

FIG. 4 is a schematic cross-sectional view schematically illustrating an example of a main configuration of a gas sensor 1B according to a second modification. In FIG. 4, the horizontal direction on the sheet surface is the axial direction (longitudinal direction) of the gas sensor 1B, the left side on the sheet surface is the distal end side, and the right side on the sheet surface is the rear end side. Similarly to the gas sensor 1A, the gas sensor 1B illustrated in FIG. 4 includes the hollow member 70A in order to prevent the ceramic housing 60 from moving to the distal end side in the axial direction while suppressing the risk of damaging the members constituting the gas sensor 1B.

Specifically, similarly to the gas sensor 1A, the gas sensor 1B includes the sensor element 10, the main metal fitting 21, the terminal metal fitting 30, the ceramic housing 60, the hollow member 70A, and the elastic body 50, and further includes the outer tube 22B similar to the outer tube 22, 22A described above. However, the gas sensor 1B further includes a spacer 100 in addition to the above-described members similar to the gas sensor 1 or the gas sensor 1A. In the gas sensor 1B, the movement of the ceramic housing 60 toward the rear end side is restricted not by the elastic body 50 or the outer tube 22A, but by the spacer 100 disposed between the ceramic housing 60 and the elastic body 50. Since the gas sensor 1B is similar to the gas sensors 1 and 1A described so far except for the above point, detailed description of components other than the outer tube 22B and the spacer 100 is omitted in the following description.

### (Outer Tube)

The outer tube 22B is different from the outer tube 22, 22A in that “the spacer 100 is stored therein”. The outer tube 22B is different from the outer tube 22 in that a hollow member 70A is accommodated instead of the hollow member 70. Furthermore, the outer tube 22B is different from the outer tube 22A in that the inner peripheral surface thereof is spaced apart from the ceramic housing 60 (not in contact with the ceramic housing 60). In other respects, the outer tube 22B is similar to the outer tube 22, 22A. For example, the outer tube 22B is a tubular (for example, a cylindrical shape) member that accommodates the rear end of the sensor element 10, the hollow member 70A, the ceramic housing 60 (terminal metal fitting 30), and the spacer 100 therein, and is, for example, a metal tubular member. The outer tube 22B is attached to a portion on the rear end side of the main metal fitting 21, and an open end on the rear end side is sealed by the elastic body 50.

### (Spacer)

The spacer 100 is an example of the “spacer” of the present invention. The spacer 100 is disposed between the ceramic housing 60 and the elastic body 50 in the axial direction of the gas sensor 1B (sensor element 10). That is, the spacer 100 is sandwiched (interposed) between the ceramic housing 60 and the elastic body 50 inside the tubular body 20 (outer tube 22B). Specifically, the spacer 100 is in contact with the rear end side of the ceramic housing 60 on the distal end side, and is in contact with the distal end side of the elastic body 50 on the rear end side. Therefore, in the gas sensor 1B, the movement of the spacer 100 toward the rear end side is restricted by the elastic body 50, and the movement of the ceramic housing 60 toward the rear end side is restricted by the spacer 100. Therefore, in the gas sensor 1B, the hollow member 70A can prevent movement of the gas sensor 1B to the distal end side of the ceramic housing 60, and the spacer 100 can prevent movement of the gas sensor 1B to the rear end side of the ceramic housing 60.

In the gas sensor 1B, the elastic body 50 is disposed on the rear end side in the axial direction with respect to the ceramic housing 60 and the spacer 100. Therefore, the gas sensor 1B has an effect that the ceramic housing 60 and the spacer 100 effectively prevent the heat generated by the heat source on the distal end side of the gas sensor 1B from being transferred to the elastic body 50. The spacer 100 disposed on the distal end side with respect to the elastic body 50 in the axial direction is preferably formed of a heat-resistant material. By forming the spacer 100 of the heat-resistant material, it is possible to prevent a situation in which the spacer 100 disposed on the distal end side with respect to the elastic body 50 in the axial direction is eroded due to heat generated by the heat source.

A lead wire 40 is inserted into the spacer 100 illustrated in FIG. 4. In the gas sensor 1B, the lead wire 40 is inserted into, for example, a through hole (not illustrated) continuously provided in the elastic body 50 and the spacer 100. Specifically, the lead wire 40 and the terminal metal fitting 30 (in particular, a lead wire holding portion that crimps and holds the lead wire 40) are accommodated inside the spacer 100. For example, a through hole extending in the axial direction is formed inside the spacer 100. Similarly to the inside of the elastic body 50, a plurality of through holes extending in the axial direction may be formed inside the spacer 100. The lead wire 40 and the lead wire holding portion of the terminal metal fitting 30 are accommodated (inserted) in the through hole formed inside the spacer 100. For example, each of the plurality of lead wires 40 and the lead wire holding portion of the terminal metal fitting 30 is accommodated (inserted) in each of the plurality of through holes formed inside the spacer 100. FIG. 4 illustrates an example in which two through holes are formed inside the spacer 100, and each of the two lead wires 40 and each of the lead wire holding portions of the two terminal metal fittings 30 are accommodated in each of the two through holes. In the gas sensor 1B, the lead wire 40 and the lead wire holding portion of the terminal metal fitting 30 are electrically connected in the spacer 100.

As described above, the spacer 100 is formed of, for example, a heat-resistant material, and is disposed between the elastic body 50 and the ceramic housing 60. By interposing the spacer 100 between the elastic body 50 and the ceramic housing 60, it is possible to prevent excessive temperature rise of the elastic body 50 at the time of using the gas sensor 1B or the like. Therefore, from the viewpoint of suppressing heat transfer to the elastic body 50, the thermal conductivity of the spacer 100 is desirably low. However, while the temperature rise of the elastic body 50 is suppressed by the spacer 100, the temperature of the spacer 100 becomes high, so that the spacer 100 itself needs to have sufficient heat resistance. Therefore, by forming the spacer 100 of a heat resistant material, it is possible to prevent the occurrence of a situation in which the spacer 100 itself is damaged by the heat generated by the heat source while suppressing the heat transfer from the heat source to the elastic body 50. As an example, the spacer 100 may be formed of ceramics. Ceramics have excellent heat resistance and generally have a higher melting point than resins. However, it is not essential for the gas sensor 1B that the spacer 100 is formed of ceramics, and the gas sensor 1B may appropriately use a material having heat resistance as the material of the spacer 100.

Although FIG. 4 illustrates an example in which the spacer 100 is one member, the spacer 100 may be configured by a plurality of constituent elements (constituent members). For example, the spacer 100 may have a spacer distal end side portion disposed on the distal end side in the axial direction and a spacer rear end side portion disposed on the rear end side. That is, the spacer 100 may have a multistage configuration (for example, a two-stage configuration) including a spacer distal end side portion and a spacer rear end side portion.

When the spacer 100 is used to suppress the temperature rise of the elastic body 50 during the use of the gas sensor 1B, the spacer distal end side portion and the spacer rear end side portion may be configured as follows. That is, as the material of the spacer distal end side portion disposed on the distal end side in the axial direction, ceramics having a melting point higher than that of the resin is selected from the viewpoint of having more excellent heat resistance than the spacer rear end side portion. Preferably, a ceramic having a thermal conductivity of 32 W/m·K or less, which is suitable also from the viewpoint of heat resistance and heat insulating properties, is selected, and more preferably, alumina (thermal conductivity: 32 W/m·K) or steatite (thermal conductivity: 2 W/m·K) is selected. On the other hand, as the material of the spacer rear end side portion in contact with the elastic body 50, a resin is selected from ceramics and the like from the viewpoint of having low thermal conductivity. Preferably, the resin used for the spacer rear end side portion is PTFE (polytetrafluoroethylene (melting point: 327° C.)) or PFA (perfluoroalkoxy alkane, melting point: 310° C.) which is a fluororesin. These resins have not only low thermal conductivity but also higher heat resistance than the rubber elastic body 50. For example, PTFE has a thermal conductivity of 0.2 W/m·K, and a continuous maximum use temperature (the maximum temperature when use at the maximum temperature continues) of 260° C.

### [Features]

As described above, the gas sensor (1, 1A, 1B) according to the present embodiment includes the sensor element 10, the main metal fitting 21, the outer tube (22, 22A, 22B), the terminal metal fitting 30, the ceramic housing 60, the elastic body 50, and the hollow member (70, 70A). The sensor element 10 extends in the axial direction and has the connector electrode 12 on the rear end side. The main metal fitting 21 is tubular, and the sensor element 10 penetrates the inside thereof in the axial direction. The outer tube is a tubular member extending in the axial direction, and attached to the outer peripheral surface on the rear end side of the main metal fitting 21. The terminal metal fitting 30 extends in the axial direction and has an element contact portion 31 on the distal end side, and the element contact portion 31 is electrically connected to the connector electrode 12 of the sensor element 10. The ceramic housing 60 is disposed inside the outer tube and accommodates the connector electrode 12 of the sensor element 10 and the element contact portion 31 of the terminal metal fitting 30. The elastic body 50 is disposed to seal the open end on the rear end side of the outer tube. The hollow member is disposed inside the outer tube, and the sensor element 10 penetrates the inside in the axial direction. The hollow member is in contact with the rear end side of the main metal fitting 21 on the distal end side in the axial direction, and is in contact with the distal end side of the ceramic housing 60 on the rear end side in the axial direction. The hollow member is formed of a material having a Young's modulus in a range from 193 to 206 GPa.

With this configuration, in the gas sensor according to the present embodiment, the hollow member (70, 70A) is in contact with the rear end side of the main metal fitting 21 on the distal end side in the axial direction, that is, the main metal fitting 21 restricts the movement to the distal end side. The ceramic housing 60 is in contact with the hollow member restricted in movement to the distal end side. Therefore, in the gas sensor according to the present embodiment, the movement of the ceramic housing 60 to the distal end side is restricted by the hollow member, that is, the hollow member prevents the ceramic housing 60 from moving to the distal end side.

In the gas sensor according to the present embodiment, the Young's modulus of the material constituting the hollow member (70, 70A) is in a range from 193 to 206 GPa.

In the first test described later, the present inventors have confirmed that the following two effects can be realized by setting the Young's modulus of the material constituting the hollow member to be in a range from 193 to 206 GPa. First, the present inventors confirmed that by setting the Young's modulus of the material constituting the hollow member to be in a range from 193 to 206 GPa, the occurrence of damage and the like in the ceramic housing 60 in contact with the hollow member can be remarkably suppressed. Secondly, the present inventors confirmed that by setting the Young's modulus of the material constituting the hollow member to be in a range from 193 to 206 GPa, the hollow member can reliably (stably) prevent the movement of the ceramic housing 60 toward the distal end side.

Therefore, the hollow member formed of a material having a Young's modulus in a range from 193 to 206 GPa can reliably prevent movement of the ceramic housing 60 toward the distal end side while remarkably suppressing occurrence of damage or the like in the ceramic housing 60.

Therefore, the gas sensor according to the present embodiment has an effect of preventing the ceramic housing 60 from moving toward the distal end side in the axial direction while suppressing the risk of breakage of a member constituting the gas sensor.

### [Modifications]

Although the embodiment of the present invention has been described above, the above description of the embodiment is merely an example of the present invention in all respects. Various improvements and modifications may be made to the above embodiments. With respect to each constituent element of the above embodiment, omission, replacement, and addition of the constituent element may be appropriately performed. In addition, the shape and dimension of each component of the above embodiment may be appropriately changed according to the embodiment. For example, the following modifications are possible. In the following description, the same reference numerals are used for the same components as those of the above embodiment, and the description of the same points as those of the above embodiment is appropriately omitted. The following modifications can be appropriately combined.

### <Shape of Ceramic Housing>

So far, an example has been described in which the ceramic housing 60 includes a plurality of (for example, the axial direction is orthogonal to the axial direction) surfaces inclined with respect to the axial direction (for example, a first distal end surface 61 and a second distal end surface 62 illustrated in FIG. 2 are included) in the end surface on the distal end side in the axial direction. However, in the gas sensor according to one aspect of the present invention, the ceramic housing 60 only needs to include one or more surfaces inclined with respect to the axial direction (for example, the axial direction is orthogonal to the axial direction) on the end surface on the distal end side in the axial direction. For example, the entire end surface on the distal end side of the ceramic housing 60 may be orthogonal to the axial direction, that is, the entire end surface on the distal end side of the ceramic housing 60 may be parallel to the radial direction. The hollow member (for example, hollow members 70, 70A) according to an aspect of the present invention may be in contact with any one of one or more “surface inclined with respect to axial direction (for example, the axial direction is orthogonal to the axial direction)” included in the end surface on the distal end side of the ceramic housing 60 on the rear end side in the axial direction.

### <Appearance of Hollow Member>

A hollow member having a cylindrical (columnar) appearance has been described as a hollow member (for example, hollow members 70, 70A) according to an aspect of the present invention. However, it is not essential that the hollow member according to one aspect of the present invention has a cylindrical appearance, and the hollow member according to one aspect of the present invention may have, for example, a triangular tubular appearance or a square tubular (quadrangular tubular shape, pentagonal tubular shape, hexagonal tubular shape, and the like) appearance. A hollow member according to an aspect of the present invention is, for example, a hollow member having a tubular shape as a whole.

### EXAMPLES

### <First Test>

In order to verify the effects of the present invention (in particular, the impact resistance performance of the gas sensor), the present inventors fabricated a gas sensor according to the following level (Example) 1 to 6, and performed an impact test on a plurality of gas sensors according to each level. However, the present invention is not limited to the following levels (Examples).

TABLE 1

| | Young's | Plate | | | | | | |
|---|---|---|---|---|---|---|---|---|
| | modulus | thickness | Height [m] and cumulative number of NG (10 samples at each level) | | | | | |
| | [GPa] | [mm] | 1.0 | 1.2 | 1.4 | 1.6 | 1.8 | 2.0 |
| Level 1 | 193 | 0.3 | 0 | 0 | 0 | 1 | 3 | 6 |
| Level 2 | 200 | 0.3 | 0 | 0 | 0 | 0 | 2 | 6 |
| Level 3 | 206 | 0.3 | 0 | 0 | 0 | 0 | 2 | 5 |
| Level 4 | 72 | 0.3 | 0 | 1 | 4 | 7 | 10 | 10 |
| Level 5 | 360 | 0.3 | 2 | 3 | 3 | 10 | 10 | 10 |
| Level 6 | — | — | 1 | 3 | 3 | 9 | 10 | 10 |

Each of the gas sensors according to Levels 1 to 5 is a gas sensor having the configuration (member) illustrated in FIG. 1, that is, a gas sensor in which a hollow member (70) is disposed between a main metal fitting (21) and a ceramic housing (60). However, the gas sensors of the level 1 to 5 have different Young's moduli of the materials constituting the hollow member (for example, a metal plate).

That is, each of the gas sensors of the level 1 to 5 includes a hollow member in which the distal end side is in contact with the rear end side of the main metal fitting and the rear end side is in contact with the distal end side of the ceramic housing inside the outer tube (22). In each of the gas sensors of the level 1 to 5, the sensor element (10) penetrates the hollow member in the axial direction. The gas sensors of the level 1 to 5 are common in that the hollow member is provided, but the gas sensors of the level 1 to 5 have different Young's moduli of the materials constituting the hollow member. Specifically, in the gas sensor of Level 1, the hollow member is formed of a metal plate “having a Young's modulus of 193 [GPa] (hereinafter, the unit [GPa] is omitted), and a plate thickness of 0.3 [mm] (hereinafter, the unit [mm] is omitted)”. In the gas sensor of Level 2, the hollow member is formed of a metal plate “having a Young's modulus of 200 and a plate thickness of 0.3”, and in the gas sensor of Level 3, the hollow member is formed of a metal plate “having a Young's modulus of 206 and a plate thickness of 0.3”. In the gas sensor of level 4, the hollow member is formed of a metal plate “having a Young's modulus of 72 and a plate thickness of 0.3”, and in the gas sensor of level 5, the hollow member is formed of a metal plate “having a Young's modulus of 360 and a plate thickness of 0.3”.

On the other hand, the gas sensor of Level 6 is a conventional gas sensor as disclosed in JP 2022-173747A, that is, a gas sensor in which a hollow member is not disposed between the main metal fitting and the ceramic housing. Specifically, the gas sensor of level 6 includes a distal end side member (formed of ceramics) extending to the rear end side of a main metal fitting surrounding the sensor element. The gas sensor of level 6 prevents the separator from moving in the axial direction of the gas sensor by sandwiching the separator between the distal end side member and the rubber cap (elastic body).

The present inventors conducted an impact test for dropping a gas sensor according to each of the levels 1 to 6 from a predetermined height (drop height) in order to compare the impact resistance performance of the gas sensor. That is, the drop heights were set to 1.0 [m] (hereinafter, the unit [m] is omitted), 1.2, 1.4, 1.6, 1.8, and 2.0, and an impact test was performed in which the gas sensors according to the respective levels 1 to 6 were dropped from the respective drop heights. Then, the present inventors confirmed whether the gas sensor after the impact test had problems such as conduction failure (for example, a contact deviation between the connector electrode (12) of the sensor element and the element contact portion (31) of the terminal metal fitting (30), or the like) and breakage (sink mark) of a member constituting the gas sensor. That is, the above-described impact test was performed on a plurality of (ten in the test) gas sensors according to Level 1, and after the impact test was performed, it was confirmed whether problems such as conduction failure and member breakage occurred in each of the plurality of gas sensors. Similarly, the above-described impact test was performed on the plurality of gas sensors related to each of the levels 2 to 6, and after the impact test was performed, it was confirmed whether problems such as conduction failure and member breakage occurred in each of the plurality of gas sensors. Referring the case where problems such as conduction failure and member breakage occurred as “NG (failure)”, the number of gas sensors determined to be NG (the cumulative number of NGs) for each drop height is summarized in Table 1 for each of the ten gas sensors at each level.

As shown in Table 1, for the ten gas sensors according to Level 1, there was no gas sensor in which problems such as conduction failure and member breakage occurred while the drop height was 1.0 to 1.4 (the cumulative NG number was 0). Among the ten gas sensors according to Level 1, the number of NG gas sensors (cumulative NG number) was 1 when the drop height was 1.6, 3 when the drop height was 1.8, and 6 when the drop height was 2.0.

For the ten gas sensors according to Level 2, there was no gas sensor in which problems such as conduction failure and member breakage occurred while the drop height was 1.0 to 1.6 (the cumulative NG number was 0). Among the ten gas sensors according to Level 2, the number of NG gas sensors (cumulative NG number) was 2 when the drop height was 1.8, and 6 when the drop height was 2.0.

For the ten gas sensors according to Level 3, there was no gas sensor in which problems such as conduction failure and member breakage occurred while the drop height was 1.0 to 1.6 (the cumulative NG number was 0). Among the ten gas sensors according to Level 3, the number of NG gas sensors (cumulative NG number) was 2 when the drop height was 1.8, and 5 when the drop height was 2.0.

For the ten gas sensors according to Level 4, when the drop height was 1.0, there was no gas sensor in which problems such as conduction failure and member breakage occurred (the cumulative NG number was 0). Among the ten gas sensors according to Level 4, the number of NG gas sensors (cumulative NG number) was 1 when the drop height was 1.2, 4 when the drop height was 1.4, 7 when the drop height was 1.6, and 10 when the drop height was 1.8 and 2.0.

Among the ten gas sensors according to Level 5, the number of NG gas sensors (cumulative NG number) was 2 when the drop height was 1.0, 3 when the drop height was 1.2 and 1.4, and 10 when the drop height was 1.6 or more.

Among the ten gas sensors according to Level 6, the number of NG gas sensors (cumulative NG number) was 1 when the drop height was 1.0, 3 when the drop height was 1.2 and 1.4, 9 when the drop height was 1.6, and 10 when the drop height was 1.8 or more.

Therefore, the present inventors confirmed the following events by the impact test and the test result (evaluation) described so far. First, the present inventors have confirmed that the gas sensors according to Levels 1 to 3 have significantly higher impact resistance performance than the gas sensor according to Level 4. That is, the gas sensors according to Levels 1 to 3 in which the Young's modulus of the material constituting the hollow member was 193 or more had significantly higher impact resistance performance than the gas sensor according to Level 4 in which the Young's modulus of the material was less than 193 (specifically, 72). When the Young's modulus of the material constituting the hollow member is less than 193, the hollow member is easily deformed or damaged, and it is difficult to reliably (stably) prevent the hollow member from moving to the distal end side of the ceramic housing, so that the impact resistance performance of the gas sensor is deteriorated. For the impact resistance performance of the gas sensor, in particular, in order to reliably prevent movement of the ceramic housing to the distal end side, it is required that “the hollow member has sufficient hardness”. The present inventors have confirmed that when the Young's modulus of the material constituting the hollow member is 193 or more, the hollow member can reliably prevent the ceramic housing from moving to the distal end side, and the impact resistance performance of the gas sensor can be remarkably improved. Therefore, by setting the Young's modulus of the material constituting the hollow member to 193 or more, the impact resistance performance of the gas sensor can be remarkably improved.

Secondly, the present inventors have confirmed that the gas sensors according to Levels 1 to 3 have significantly higher impact resistance performance than the gas sensor according to Level 5. That is, the gas sensors according to Levels 1 to 3 in which the Young's modulus of the material constituting the hollow member was 206 or less had significantly higher impact resistance performance than the gas sensor according to Level 5 in which the Young's modulus of the material was larger than 206 (specifically, 360). Therefore, by setting the Young's modulus of the material constituting the hollow member to 206 or less, the impact resistance performance of the gas sensor can be remarkably improved.

Third, the present inventors have confirmed that the gas sensors according to Levels 1 to 3 have significantly higher impact resistance performance than the gas sensor according to Level 6. That is, the gas sensors of Levels 1 to 3 including the hollow member between the main metal fitting and the ceramic housing had significantly higher impact resistance performance than the gas sensor of Level 6 (conventional gas sensor) not including the hollow member. Therefore, the impact resistance performance of the gas sensor can be remarkably improved by including the hollow member disposed between the main metal fitting and the ceramic housing.

As described above, the present inventors have confirmed that the following effects can be realized by disposing a hollow member between the main metal fitting and the ceramic housing, particularly by setting the Young's modulus of the material constituting the hollow member to 193 or more and 206 or less. That is, the present inventors have confirmed that the impact resistance performance of the gas sensor can be significantly improved by disposing a hollow member formed of a material having a Young's modulus of 193 or more and 206 or less between the main metal fitting and the ceramic housing.

### <Second Test>

In order to verify an additional effect (heat resistance of the gas sensor) of the present invention, the present inventors fabricated a gas sensor according to the following level (Example) 7 to 11, and performed a thermal load test on a plurality of gas sensors according to each level. However, the present invention is not limited to the following levels (Examples).

TABLE 2

| | Young's modulus | Plate thickness | TC temperature |
|---|---|---|---|
| | [GPa] | [mm] | [° C.] |
| Level 7 | 200 | 0.3 | 283.9 |
| Level 8 | 200 | 0.5 | 286.5 |
| Level 9 | 200 | 0.8 | 292.7 |
| Level 10 | 200 | 1.2 | 315.7 |
| Level 11 | — | — | 317.1 |

Each of the gas sensors according to Levels 7 to 10 is a gas sensor having the configuration (member) illustrated in FIG. 1, that is, a gas sensor in which a hollow member is disposed between a main metal fitting and a ceramic housing. The hollow member included in each of the gas sensors according to Levels 7 to 10 is entirely formed of a material having a Young's modulus in a range from 193 to 206 GPa. However, the gas sensors of the level 7 to 10 have different plate thicknesses of the materials constituting the hollow member (for example, a metal plate).

That is, each of the gas sensors of the level 7 to 10 includes a hollow member in which the distal end side is in contact with the rear end side of the main metal fitting and the rear end side is in contact with the distal end side of the ceramic housing inside the outer tube. In each of the gas sensors of the level 7 to 10, the sensor element penetrates the hollow member in the axial direction. The gas sensors of the level 7 to 10 are common in that the hollow member is provided, but the gas sensors of the level 7 to 10 are different from each other in the plate thickness of the material constituting the hollow member. Specifically, in the gas sensor of level 7, the hollow member is formed of a metal plate “having a Young's modulus of 200 and a plate thickness of 0.3”. In the gas sensor of level 8, the hollow member is formed of a metal plate “having a Young's modulus of 200 and a plate thickness of 0.5”, and in the gas sensor of level 9, the hollow member is formed of a metal plate “having a Young's modulus of 200 and a plate thickness of 0.8”. In the gas sensor of level 10, the hollow member is made of a metal plate “having the Young's modulus of 200 and the plate thickness of 1.2”.

On the other hand, the gas sensor of level 11 is a conventional gas sensor as disclosed in JP 2022-173747A, and is a gas sensor similar to the gas sensor of level 6 in Table 1.

The present inventors conducted a thermal load test for heating a HEX (main metal fitting) to about 440 [° C.] (hereinafter, the unit [° C.] is omitted) in order to compare the heat resistance of the gas sensor according to each of the levels 7 to 11. For example, the gas sensor according to each of the levels 7 to 11 was placed in an environment where the external temperature around the gas sensor was about 440 for a predetermined time, and the temperature of the HEX was set to about 440. The present inventors measured (confirmed) the temperature (TC temperature) of the elastic body (50) when the temperature of the HEX was about 440 for the gas sensor according to each of the levels 7 to 11. Table 2 shows the TC temperature of the elastic body (corresponding to the rubber cap of the conventional gas sensor) when the temperature of the HEX is about 440 for the gas sensor according to each level. The elastic body may be eroded when the temperature increases, but when erosion occurs, the reference gas (reference air) in the outer tube (22) may be contaminated or the seismic resistance of the gas sensor may deteriorate. In addition, an electrode (for example, a reference electrode provided so as to be contactable with a reference gas) included in the sensor element may be contaminated by an organic gas accompanying decomposition of the elastic body by heat. As the temperature of the elastic body is lower, it is possible to prevent the above-described contamination of the reference gas and the reference electrode, deterioration of the seismic resistance of the gas sensor, and the like.

As shown in Table 2, for the gas sensor according to each of the levels 7 to 11, the TC temperatures of the elastic body 50 when the temperature of the HEX was about 440 were 283.9, 286.5, 292.7, 315.7, and 317.1.

Therefore, the present inventors confirmed the following events from the above-described heat load test and the test result (measurement result). First, the present inventors have confirmed that the temperature (TC temperature) of the elastic body of each of the gas sensors according to Levels 7 to 10 is lower than that of the gas sensor according to Level 11, that is, the gas sensor has high heat resistance. That is, the gas sensors according to Levels 7 to 10 including the hollow member between the main metal fitting and the ceramic housing can effectively suppress heat transfer to the elastic body and have high heat resistance as compared with the gas sensor (conventional gas sensor) according to Level 11 not including the hollow member.

Secondly, the present inventors have confirmed that the temperature of the elastic body of each of the gas sensors according to Levels 7 to 9 is significantly lower than that of the gas sensor according to Level 10, that is, the heat resistance is significantly higher. That is, the gas sensors according to Levels 7 to 9 in which the plate thickness of the material constituting the hollow member was 0.8 or less had significantly higher heat resistance than the gas sensor according to Level 10 in which the plate thickness of the material was larger than 0.8 (specifically, 1.2). Therefore, the heat resistance of the gas sensor can be remarkably improved by setting the plate thickness of the material constituting the hollow member to 0.8 or less. In the gas sensor, the heat resistance can be improved by disposing the hollow member between the main metal fitting and the ceramic housing, and in particular, the heat resistance can be remarkably improved by setting the plate thickness of the material constituting the hollow member to 0.8 or less.

It is not realistic to configure the hollow member using a material (for example, a metal plate having a plate thickness of less than 0.3) having a plate thickness of less than 0.3 in consideration of mass productivity and the like. As described above, in the gas sensor, the hollow member prevents the ceramic housing from moving toward the distal end side. However, when the thickness of the material constituting the hollow member is less than 0.3, the hollow member may fail to prevent the ceramic housing from moving toward the distal end side. That is, when the plate thickness of the material constituting the hollow member is less than 0.3, it may be difficult for the hollow member to have sufficient strength to prevent the movement of the ceramic housing toward the distal end side. Therefore, the plate thickness of the material constituting the hollow member is desirably 0.3 or more.

As described above, the present inventors have confirmed that the following effects can be realized by disposing a hollow member between the main metal fitting and the ceramic housing, particularly by setting the plate thickness of the material constituting the hollow member to 0.3 or more and 0.8 or less. That is, the present inventors have confirmed that the heat resistance of the gas sensor can be remarkably improved by disposing a hollow member formed of a material having a plate thickness of 0.3 or more and 0.8 or less between the main metal fitting and the ceramic housing.

### REFERENCE SIGNS LIST

1, 1A, 1B Gas sensor
10 Sensor element
12 Connector electrode
21 Main metal fitting
22, 22A, 22B Outer tube
30 Terminal metal fitting
31 Element contact portion
50 Elastic body
60 Ceramic housing
70, 70A Hollow member
100 Spacer

1. A gas sensor comprising:
a sensor element extending in an axial direction and having a connector electrode on a rear end side;
a tubular main metal fitting in which the sensor element penetrates the inside in the axial direction;
a tubular outer tube extending in the axial direction and attached to an outer peripheral surface on a rear end side of the main metal fitting;
a terminal metal fitting extending in the axial direction and including an element contact portion electrically connected to the connector electrode on a distal end side;
a ceramic housing accommodating the connector electrode and the element contact portion and disposed inside the outer tube;
an elastic body disposed to seal an open end on a rear end side of the outer tube; and
a hollow member including a material having a Young's modulus in a range from 193 to 206 GPa and disposed inside the outer tube, in which
the sensor element penetrates the inside in the axial direction,
a distal end side in the axial direction is in contact with the rear end side of the main metal fitting, and
a rear end side in the axial direction is in contact with a distal end side of the ceramic housing.

2. The gas sensor according to claim 1, wherein the hollow member is formed of a thin plate having a thickness in a range from 0.3 to 0.8 mm.

3. The gas sensor according to claim 1, wherein the hollow member includes a plurality of members.

4. The gas sensor according to claim 1, wherein the hollow member and the main metal fitting are integrally formed.

5. The gas sensor according to claim 1, wherein the ceramic housing is spaced apart from the elastic body.

6. The gas sensor according to claim 1, wherein the main metal fitting includes a surface orthogonal to the axial direction on the rear end side, and
the hollow member is in contact with the surface of the main metal fitting orthogonal to the axial direction.

7. The gas sensor according to claim 1, wherein the hollow member is spaced apart from the sensor element.

8. The gas sensor according to claim 1, further comprising a spacer disposed between the ceramic housing and the elastic body in the axial direction.

9. The gas sensor according to claim 2, wherein the hollow member includes a plurality of members.

10. The gas sensor according to claim 2, wherein the hollow member and the main metal fitting are integrally formed.

11. The gas sensor according to claim 2, wherein the ceramic housing is spaced apart from the elastic body.

12. The gas sensor according to claim 2, wherein the main metal fitting includes a surface orthogonal to the axial direction on the rear end side, and
the hollow member is in contact with the surface of the main metal fitting orthogonal to the axial direction.

13. The gas sensor according to claim 2, wherein the hollow member is spaced apart from the sensor element.

14. The gas sensor according to claim 2, further comprising a spacer disposed between the ceramic housing and the elastic body in the axial direction.
